SEQUENCE LISTING (43 sequences)

SEQ ID NO: 1 - protein, 133 aa, enterobacteria phage Qbeta 39803
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - DNA, 402 bp, enterobacteria phage Qbeta 39803
atggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatacgctgacgtgaccttttcgttcacgcagtatagtaccgatgaggaacgagcttttgttcgtacagagcttgctgctctgctcgctagtcctctgctgatcgatgctattgatcagctgaacccagcgtattaa

SEQ ID NO: 3 - RNA, 561 nt, Artificial Sequence
  Synthesized
ggggaauugugagcggauaacaauuccccucuagagguuuaaucagaguagaggagcugacuccuuugguuggacuaggauccaauaauuuuguuuaacuuuaagaaggagauauaccauggcaaaauuagagacuguuacuuuagguaacaucgggaaagauggaaaacaaacucugguccucaauccgcgugggguaaaucccacuaacggcguugccucgcuuucacaagcgggugcaguuccugcgcuggagaagcguguuaccguuucgguaucucagccuucucgcaaucguaagaacuacaagguccagguuaagauccagaacccgaccgcuugcacugcaaacgguucuugugacccauccguuacucgccaggcauaugcugacgugaccuuuucguucacgcaguauaguaccgaugaggaacgagcuuuuguucguacagagcuugcugcucugcucgcuaguccucugcugaucgaugcuauugaucagcugaacccagcguauugauaaggaugaaaugcaugucuaagacagcaucugcagaauaa

SEQ ID NO: 4 - protein, 258 aa, Escherichia coli
  Rev_tag (1)..(29)
  Peptidase_E (30)..(258)
MELTRQARRNRRRRWRERQRGSGGELSAAMELLLLSNSTLPGKAWLEHALPLIAEQLQGRRSAVFIPFAGVTQTWDDYTAKTAAVLAPLGVSVTGIHSVVDPVAAIENAEIVIVGGGNTFQLLKQCRERGLLAPITDVVKRGALYIGWSAGANLACPTIRTTNDMPIVDPQGFDALNLFPLQINPHFTNALPEGHKGETREQRIRELLVVAPELTIIGLPEGNWITVSKGHATLGGPNTTYVFKAGEEAVPLEAGHRF

SEQ ID NO: 5 - DNA, 777 bp, Escherichia coli
  Rev_tag (1)..(87)
  pepE (88)..(777)
atggagctgacaagacaggcacgtcgaaatcgcaggagacgatggcgggaacgtcaaaggggctcgggtggcgagctctcggccgccatggaactgcttttattgagtaactcgacgctgccgggtaaagcctggctggaacatgcactgccgctaattgctgaacagttgcagggtcgccgctcagcggtgtttatccctttcgctggcgtaacgcagacctgggatgattacacagcgaaaacggctgcggttctcgctccgctgggtgtttctgtcaccggtattcatagcgttgtcgatcccgttgccgcgattgaaaatgctgagatcgtgattgtcggcggcgggaatactttccagttgctgaaacagtgccgcgagcgcgggctgctggcaccaattactgacgtggttaaacgtggcgctctgtatattggctggagcgcaggcgctaaccttgcttgcccaactattcgtaccaccaacgatatgccgattgtcgatccgcaaggtttcgatgcgctaaatctgttcccgctgcaaatcaacccgcacttcaccaacgcgctgccggaaggccataaaggtgaaacccgtgagcagcgtattcgcgaactgctggtcgtcgcgccagaactgacgattattggtctaccggaaggtaactggatcacagtgagtaaaggtcacgctacgctgggtggcccgaacaccacttatgtgtttaaggctggtgaagaagcggttccgctggaagctggtcaccgtttttaa

SEQ ID NO: 6 - protein, 579 aa, Photinus pyralis
  Rev_tag (1)..(29)
  Luciferase (30)..(579)
MELTRQARRNRRRRWRERQRGSGGELSAAMEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 7 - DNA, 1740 bp, Photinus pyralis
  Rev_tag (1)..(87)
  Luc (88)..(1740)
atggagctgacaagacaggcacgtcgaaatcgcaggagacgatggcgggaacgtcaaaggggctcgggtggcgagctctcggccgccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccaaaggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 8 - protein, 579 aa, Artificial Sequence; identical to SEQ ID NO: 6
  Synthesized

SEQ ID NO: 9 - DNA, 1740 bp, Artificial Sequence; identical to SEQ ID NO: 7
  Synthesized

SEQ ID NO: 10 - protein, 187 aa, Saccharomyces cerevisiae
  Rev_tag (1)..(29)
  Cytosine_deaminase (30)..(187)
MELTRQARRNRRRRWRERQRGSGGELSAAMVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 11 - DNA, 564 bp, Artificial Sequence
  Synthesized
atggagctgacaagacaggcacgtcgaaatcgcaggagacgatggcgggaacgtcaaaggggctcgggtggcgagctctcggccgccatggttaccggtggtatggcttctaaatgggaccagaaaggtatggacatcgcttacgaagaagctgctctgggttacaaagaaggtggtgttccaattggtggttgcctgatcaacaacaaagacggttctgttctgggtcgtggtcacaacatgcgtttccagaaaggttctgctaccctgcacggtgaaatctctaccctggaaaactgcggtcgtctggaaggtaaagtttacaaagacaccaccctgtacaccaccctgtctccgtgcgacatgtgcaccggtgctatcatcatgtacggtatcccgcgttgcgttgttggtgaaaacgttaacttcaaatctaaaggtgaaaaatacctgcagacccgtggtcacgaagttgttgttgttgacgacgaacgttgcaaaaaaatcatgaaacagttcatcgacgaacgtccgcaggactggttcgaagacatcggtgaataa

SEQ ID NO: 12 - protein, 187 aa, Artificial Sequence
  Synthesized
MELTRQARRNRRRRWRERQRGSGGELSAAMVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGE

SEQ ID NO: 13 - DNA, 564 bp, Artificial Sequence
  Synthesized
atggagctgacaagacaggcacgtcgaaatcgcaggagacgatggcgggaacgtcaaaggggctcgggtggcgagctctcggccgccatggttaccggtggtatggcttctaaatgggaccagaaaggtatggacatcgcttacgaagaagctctgctgggttacaaagaaggtggtgttccaattggtggttgcctgatcaacaacaaagacggttctgttctgggtcgtggtcacaacatgcgtttccagaaaggttctgctaccctgcacggtgaaatctctaccctggaaaactgcggtcgtctggaaggtaaagtttacaaagacaccaccctgtacaccaccctgtctccgtgcgacatgtgcaccggtgctatcatcatgtacggtatcccgcgttgcgttatcggtgaaaacgttaacttcaaatctaaaggtgaaaaatacctgcagacccgtggtcacgaagttgttgttgttgacgacgaacgttgcaaaaaactgatgaaacagttcatcgacgaacgtccgcaggactggttcgaagacatcggtgaataa

SEQ ID NO: 14 - protein, 457 aa, Escherichia coli
  Rev_tag (1)..(39)
  Cytosine_deaminase (30)..(457)
MELTRQARRNRRRRWRERQRGSGGELSAAMGSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 15 - DNA, 1374 bp, Escherichia coli
  Rev_tag (1)..(87)
  codA (88)..(1374)
atggagctgacaagacaggcacgtcgaaatcgcaggagacgatggcgggaacgtcaaaggggctcgggtggcgagctctcggccgccatgggctcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttaa

SEQ ID NO: 16 - protein, 245 aa, Saccharomyces cerevisiae
  Rev_tag (1)..(29)
  UPRT (30)..(245)
MELTRQARRNRRRRWRERQRGSGGELSAAMSSEPFKNVYLLPQTNQLLGLYTIIRNKNTTRPDFIFYSDRIIRLLVEEGLNHLPVQKQIVETDTNENFEGVSFMGKICGVSIVRAGESMEQGLRDCCRSVRIGKILIQRDEETALPKLFYEKLPEDISERYVFLLDPMLATGGSAIMATEVLIKRGVKPERIYFLNLICSKEGIEKYHAAFPEVRIVTGALDRGLDENKYLVPGLGDFGDRYYCV

SEQ ID NO: 17 - DNA, 741 bp, Saccharomyces cerevisiae
  Rev_tag (1)..(87)
  FUR1 (88)..(741)
atggagctgacaagacaggcacgtcgaaatcgcaggagacgatggcgggaacgtcaaaggggctcgggtggcgagctctcggccgccatgggctcttcggaaccatttaagaacgtctacttgctacctcaaacaaaccaattgctgggtttgtacaccatcatcagaaataagaatacaactagacctgatttcattttctactccgatagaatcatcagattgttggttgaagaaggtttgaaccatctacctgtgcaaaagcaaattgtggaaactgacaccaacgaaaacttcgaaggtgtctcattcatgggtaaaatctgtggtgtttccattgtcagagctggtgaatcgatggagcaaggattaagagactgttgtaggtctgtgcgtatcggtaaaattttaattcaaagggacgaggagactgctttaccaaagttattctacgaaaaattaccagaggatatatctgaaaggtatgtcttcctattagacccaatgctggccaccggtggtagtgctatcatggctacagaagtcttgattaagagaggtgttaagccagagagaatttacttcttaaacctaatctgtagtaaggaagggattgaaaaataccatgccgccttcccagaggtcagaattgttactggtgccctcgacagaggtctagatgaaaacaagtatctagttccagggttgggtgactttggtgacagatactactgtgtttaa

SEQ ID NO: 18 - protein, 403 aa, Saccharomyces cerevisiae
  Rev_tag (1)..(29)
  CD-UPRT (39)..(403)
MELTRQARRNRRRRWRERQRGSGGELSAAMVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSEPFKNVYLLPQTNQLLGLYTIIRNKNTTRPDFIFYSDRIIRLLVEEGLNHLPVQKQIVETDTNENFEGVSFMGKICGVSIVRAGESMEQGLRDCCRSVRIGKILIQRDEETALPKLFYEKLPEDISERYVFLLDPMLATGGSAIMATEVLIKRGVKPERIYFLNLICSKEGIEKYHAAFPEVRIVTGALDRGLDENKYLVPGLGDFGDRYYCV

SEQ ID NO: 19 - DNA, 810 bp, Saccharomyces cerevisiae
  Rev_tag (1)..(87)
  FCU1 (88)..(810)
atggagctgacaagacaggcacgtcgaaatcgcaggagacgatggcgggaacgtcaaaggggctcgggtggcgagctctcggccatggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactgcgcgacgtcgttatcggtatgggtgcctgcaccgattccaaagttaaccgtatccgttttaaagaccatgactttgccgctatcgctgactttgacatggtgcgtaacgcggtagacgcggctaaagcactgggtgttgatgctcgcgtgggtaacctgttctccgctgacctgttctactctccggacggcgaaatgttcgacgtgatggaaaaatacggcatcctcggcgtggaaatggaagcggctggtatctacggcgtcgctgcagagtttggcgcgaaagccctgaccatctgcaccgtgtctgaccacatccgcactcacgagcagaccactgccgctgagcgtcagaccaccttcaacgacatgatcaaaatcgcactggaatccgttctgctgggcgataaagagtaactcgag

SEQ ID NO: 20 - protein, 267 aa, Escherichia coli
  Rev_tag (1)..(29)
  PNP (30)..(267)
MELTRQARRNRRRRWRERQRGSGGELSAMATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGVDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 21 - DNA, 810 bp, Escherichia coli; identical to SEQ ID NO: 19
  Rev_tag (1)..(87)
  PNP (88)..(810)

SEQ ID NO: 22 - protein, 357 aa, Escherichia coli
  Rev_tag (1)..(29)
  AsparaginaseII (30)..(357)
MELTRQARRNRRRRWRERQRGSGGELSAAMGLPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 23 - DNA, 1074 bp, Escherichia coli
  Rev_tag (1)..(87)
  AnsB (88)..(1074)
atggagctgacaagacaggcacgtcgaaatcgcaggagacgatggcgggaacgtcaaaggggctcgggtggcgagctctcggccgccatgggcttacccaatatcaccattttagcaaccggcgggaccattgccggtggtggtgactccgcaaccaaatctaactacacagtgggtaaagttggcgtagaaaatctggttaatgcggtgccgcaactaaaagacattgcgaacgttaaaggcgagcaggtagtgaatatcggctcccaggacatgaacgataatgtctggctgacactggcgaaaaaaattaacaccgactgcgataagaccgacggcttcgtcattacccacggtaccgacacgatggaagaaactgcttacttcctcgacctgacggtgaaatgcgacaaaccggtggtgatggtcggcgcaatgcgtccgtccacgtctatgagcgcagacggtccattcaacctgtataacgcggtagtgaccgcagctgataaagcctccgccaaccgtggcgtgctggtagtgatgaatgacaccgtgcttgatggccgtgacgtcaccaaaaccaacaccaccgacgtagcgaccttcaagtctgttaactacggtcctctgggttacattcacaacggtaagattgactaccagcgtaccccggcacgtaagcataccagcgacacgccattcgatgtctctaagctgaatgaactgccgaaagtcggcattgtttataactacgctaacgcatccgatcttccggctaaagcactggtagatgcgggctatgatggcatcgttagcgctggtgtgggtaacggcaacctgtataaatctgtgttcgacacgctggcgaccgccgcgaaaaccggtactgcagtcgtgcgttcttcccgcgtaccgacgggcgctaccactcaggatgccgaagtggatgatgcgaaatacggcttcgtcgcctctggcacgctgaacccgcaaaaagcgcgcgttctgctgcaactggctctgacgcaaaccaaagatccgcagcagatccagcagatcttcaatcagtactaa

SEQ ID NO: 24 - protein, 236 aa, Escherichia coli
  Rev_tag (1)..(29)
  Superoxide_dismutase_A (30)..(236)
MELTRQARRNRRRRWRERQRGSGGELSAAMGSYTLPSLPYAYDALEPHFDKQTMEIHHTKHHQTYVNNANAALESLPEFANLPVEELITKLDQLPADKKTVLRNNAGGHANHSLFWKGLKKGTTLQGDLKAAIERDFGSVDNFKAEFEKAAASRFGSGWAWLVLKGDKLAVVSTANQDSPLMGEAISGASGFPIMGLDVWEHAYYLKFQNRRPDYIKEFWNVVNWDEAAARFAAKK

SEQ ID NO: 25 - DNA, 711 bp, Escherichia coli
  Rev_tag (1)..(87)
  SodA (88)..(711)
atggagctgacaagacaggcacgtcgaaatcgcaggagacgatggcgggaacgtcaaaggggctcgggtggcgagctctcggccgccatgggcagctataccctgccatccctgccgtatgcttacgatgccctggaaccgcacttcgataagcagacgatggaaatccaccacaccaaacaccatcagacctacgtaaacaacgccaacgcggcgctggaaagcctgccagaatttgccaacctgccggttgaagagctgatcaccaaactggaccagctgccagcagacaagaaaaccgtactgcgcaacaacgctggcggtcacgctaaccacagcctgttctggaaaggtctgaaaaaaggcaccaccctgcagggtgacctgaaagcggctatcgaacgtgacttcggctccgttgataacttcaaagcagaatttgaaaaagcggcagcttcccgctttggttccggctgggcatggctggtgctgaaaggcgataaactggcggtggtttctactgctaaccaggattctccgctgatgggtgaagctatttctggcgcttccggcttcccgattatgggcctggatgtgtgggaacatgcttactacctgaaattccagaaccgccgtccggactacattaaagagttctggaacgtggtgaactgggacgaagcagcggcacgttttgcggcgaagaaataa

SEQ ID NO: 26 - protein, 223 aa, Escherichia coli
  Rev_tag (1)..(29)
  Superoxide_dismutase_B (30)..(223)
MELTRQARRNRRRRWRERQRGSGGELSAAMGSFELPALPYAKDALAPHISAETIEYHYGKHHQTYVTNLNNLIKGTAFEGKSLEEIIRSSEGGVFNNAAQVWNHTFYWNCLAPNAGGEPTGKVAEAIAASFGSFADFKAQFTDAAIKNFGSGWTWLVKNSDGKLAIVSTSNAGTPLTTDATPLLTVDVWEHAYYIDYRNARPGYLEHFWALVNWEFVAKNLAA

SEQ ID NO: 27 - DNA, 672 bp, Escherichia coli
  Rev_tag (1)..(87)
  SodB (88)..(672)
atggagctgacaagacaggcacgtcgaaatcgcaggagacgatggcgggaacgtcaaaggggctcgggtggcgagctctcggccgccatgggctcattcgaattacctgcactaccatatgctaaagatgctctggcaccgcacatttctgcggaaaccatcgagtatcactacggcaagcaccatcagacttatgtcactaacctgaacaacctgattaaaggtaccgcgtttgaaggtaaatcactggaagagattattcgcagctctgaaggtggcgtattcaacaacgcagctcaggtctggaaccatactttctactggaactgcctggcaccgaacgccggtggcgaaccgactggaaaagtcgctgaagctatcgccgcatcttttggcagctttgccgatttcaaagcgcagtttactgatgcagcgatcaaaaactttggttctggctggacctggctggtgaaaaacagcgatggcaaactggctatcgtttcaacctctaacgcgggtactccgctgaccaccgatgcgactccgctgctgaccgttgatgtctgggaacacgcttattacatcgactatcgcaatgcacgtcctggctatctggagcacttctgggcgctggtgaactgggaattcgtagcgaaaaatctcgctgcataa

SEQ ID NO: 28 - protein, 184 aa, Escherichia coli
  Rev_tag (1)..(29)
  Superoxide_dismutase_C (30)..(184)
MELTRQARRNRRRRWRERQRGSGGELSAAMASEKVEMNLVTSQGVGQSIGSVTITETDKGLEFSPDLKALPPGEHGFHIHAKGSCQPATKDGKASAAESAGGHLDPQNTGKHEGPEGAGHLGDLPALVVNNDGKATDAVIAPRLKSLDEIKDKALMVHVGGDNMSDQPKPLGGGGERYACGVIK

SEQ ID NO: 29 - DNA, 558 bp, Escherichia coli
  Rev_tag (1)..(87)
  SodC (88)..(558)
atggagctgacaagacaggcacgtcgaaatcgcaggagacgatggcgggaacgtcaaaggggctcgggtggcgagctctcggccatggccatggccagtgaaaaagtcgagatgaacctcgtcacgtcgcaaggggtagggcagtcaattggtagcgtcaccattactgaaaccgataaaggtctggagttttcgcccgatctgaaagcattaccccccggtgaacatggcttccatattcatgccaaaggaagctgccagccagccaccaaagatggcaaagccagcgccgcggaatccgcaggcgggcatcttgatccacaaaataccggtaaacatgaagggccagaaggtgccgggcatttaggcgatctgcctgcactggtcgtcaataatgacggcaaagctaccgatgccgtcatcgcgcctcgtctgaaatcactggatgaaatcaaagacaaagcgctgatggtccacgttggcggcgataatatgtccgatcaacctaaaccgctgggcggtggcggtgaacgctatgcctgtggtgtaattaagtaa

SEQ ID NO: 30 - protein, 756 aa, Escherichia coli
  Rev_tag (1)..(29)
  Catalase (30)..(756)
MELTRQARRNRRRRWRERQRGSGGELSAAMASTSDDIHNTTATGKCPFHQGGHDQSAGAGTTTRDWWPNQLRVDLLNQHSNRSNPLGEDFDYRKEFSKLDYYGLKKDLKALLTESQPWWPADWGSYAGLFIRMAWHGAGTYRSIDGRGGAGRGQQRFAPLNSWPDNVSLDKARRLLWPIKQKYGQKISWADLFILAGNVALENSGFRTFGFGAGREDVWEPDLDVNWGDEKAWLTHRHPEALAKAPLGATEMGLIYVNPEGPDHSGEPLSAAAAIRATFGNMGMNDEETVALIAGGHTLGKTHGAGPTSNVGPDPEAAPIEEQGLGWASTYGSGVGADAITSGLEVVWTQTPTQWSNYFFENLFKYEWVQTRSPAGAIQFEAVDAPEIIPDPFDPSKKRKPTMLVTDLTLRFDPEFEKISRRFLNDPQAFNEAFARAWFKLTHRDMGPKSRYIGPEVPKEDLIWQDPLPQPIYNPTEQDIIDLKFAIADSGLSVSELVSVAWASASTFRGGDKRGGANGARLALMPQRDWDVNAAAVRALPVLEKIQKESGKASLADIIVLAGVVGVEKAASAAGLSIHVPFAPGRVDARQDQTDIEMFELLEPIADGFRNYRARLDVSTTESLLIDKAQQLTLTAPEMTALVGGMRVLGANFDGSKNGVFTDRVGVLSNDFFVNLLDMRYEWKATDESKELFEGRDRETGEVKFTASREDLVFGSNSVLRAVAEVYASSDAHEKFVKDFVAAWVKVMNLDRFDLL

SEQ ID NO: 31 - DNA, 2271 bp, Escherichia coli
  Rev_tag (1)..(87)
  KatG (88)..(2271)
atggagctgacaagacaggcacgtcgaaatcgcaggagacgatggcgggaacgtcaaaggggctcgggtggcgagctctcggccgccatggcaagcacgtcagacgatatccataacaccacagccactggcaaatgcccgttccatcagggcggtcacgaccagagtgcgggggcgggcacaaccactcgcgactggtggccaaatcaacttcgtgttgacctgttaaaccaacattctaatcgttctaacccactgggtgaggactttgactaccgcaaagaattcagcaaattagattactacggcctgaaaaaagatctgaaagccctgttgacagaatctcaaccgtggtggccagccgactggggcagttacgccggtctgtttattcgtatggcctggcacggcgcggggacttaccgttcaatcgatggacgcggtggcgcgggtcgtggtcagcaacgttttgcaccgctgaactcctggccggataacgtaagcctcgataaagcgcgtcgcctgttgtggccaatcaaacagaaatatggtcagaaaatctcctgggccgacctgtttatcctcgcgggtaacgtggcgctagaaaactccggcttccgtaccttcggttttggtgccggtcgtgaagacgtctgggaaccggatctggatgttaactggggtgatgaaaaagcctggctgactcaccgtcatccggaagcgctggcgaaagcaccgctgggtgcaaccgagatgggtctgatttacgttaacccggaaggcccggatcacagcggcgaaccgctttctgcggcagcagctatccgcgcgaccttcggcaacatgggcatgaacgacgaagaaaccgtggcgctgattgcgggtggtcatacgctgggtaaaacccacggtgccggtccgacatcaaatgtaggtcctgatccagaagctgcaccgattgaagaacaaggtttaggttgggcgagcacttacggcagcggcgttggcgcagatgccattacctctggtctggaagtagtctggacccagacgccgacccagtggagcaactatttcttcgagaacctgttcaagtatgagtgggtacagacccgcagcccggctggcgcaatccagttcgaagcggtagacgcaccggaaattatcccggatccgtttgatccgtcgaagaaacgtaaaccgacaatgctggtgaccgacctgacgctgcgttttgatcctgagttcgagaagatctctcgtcgtttcctcaacgatccgcaggcgttcaacgaagcctttgcccgtgcctggttcaaactgacgcacagggatatggggccgaaatctcgctacatcgggccggaagtgccgaaagaagatctgatctggcaagatccgctgccgcagccgatctacaacccgaccgagcaggacattatcgatctgaaattcgcgattgcggattctggtctgtctgttagtgagctggtatcggtggcctgggcatctgcttctaccttccgtggtggcgacaaacgcggtggtgccaacggtgcgcgtctggcattaatgccgcagcgcgactgggatgtgaacgccgcagccgttcgtgctctgcctgttctggagaaaatccagaaagagtctggtaaagcctcgctggcggatatcatagtgctggctggtgtggttggtgttgagaaagccgcaagcgccgcaggtttgagcattcatgtaccgtttgcgccgggtcgcgttgatgcgcgtcaggatcagactgacattgagatgtttgagctgctggagccaattgctgacggtttccgtaactatcgcgctcgtctggacgtttccaccaccgagtcactgctgatcgacaaagcacagcaactgacgctgaccgcgccggaaatgactgcgctggtgggcggcatgcgtgtactgggtgccaacttcgatggcagcaaaaacggcgtcttcactgaccgcgttggcgtattgagcaatgacttcttcgtgaacttgctggatatgcgttacgagtggaaagcgaccgacgaatcgaaagagctgttcgaaggccgtgaccgtgaaaccggcgaagtgaaatttacggccagccgtgaggatctggtgtttggttctaactccgtcctgcgtgcggtggcggaagtttacgccagtagcgatgcccacgagaagtttgttaaagacttcgtggcggcatgggtgaaagtgatgaacctcgaccgtttcgacctgctgtaa

SEQ ID NO: 32 - protein, 289 aa, Escherichia coli
  Rev_tag (1)..(29)
  Deoxyribose-phosphate_aldolase (30)..(289)
MELTRQARRNRRRRWRERQRGSGGELSAAMGTDLKASSLRALKLMDLTTLNDDDTDEKVIALCHQAKTPVGNTAAICIYPRFIPIARKTLKEQGTPEIRIATVTNFPHGNDDIDIALAETRAAIAYGADEVDVVFPYRALMAGNEQVGFDLVKACKEACAAANVLLKVIIETGELKDEALIRKASEISIKAGADFIKTSTGKVAVNATPESARIMMEVIRDMGVEKTVGFKPAGGVRTAEDAQKYLAIADELFGADWADARHYRFGASSLLASLLKALGHGDGKSASSY

SEQ ID NO: 33 - DNA, 870 bp, Escherichia coli
  Rev_tag (1)..(87)
  DeoC (88)..(870)
atggagctgacaagacaggcacgtcgaaatcgcaggagacgatggcgggaacgtcaaaggggctcgggtggcgagctctcggccgccatgggcactgatctgaaagcaagcagcctgcgtgcactgaaattgatggacctgaccaccctgaatgacgacgacaccgacgagaaagtgatcgccctgtgtcatcaggccaaaactccggtcggcaataccgccgctatctgtatctatcctcgctttatcccgattgctcgcaaaactctgaaagagcagggcaccccggaaatccgtatcgctacggtaaccaacttcccacacggtaacgacgacatcgacatcgcgctggcagaaacccgtgcggcaatcgcctacggtgctgatgaagttgacgttgtgttcccgtaccgcgcgctgatggcgggtaacgagcaggttggttttgacctggtgaaagcctgtaaagaggcttgcgcggcagcgaatgtactgctgaaagtgatcatcgaaaccggcgaactgaaagacgaagcgctgatccgtaaagcgtctgaaatctccatcaaagcgggtgcggacttcatcaaaacctctaccggtaaagtggctgtgaacgcgacgccggaaagcgcgcgcatcatgatggaagtgatccgtgatatgggcgtagaaaaaaccgttggtttcaaaccggcgggcggcgtgcgtactgcggaagatgcgcagaaatatctcgccattgcagatgaactgttcggtgctgactgggcagatgcgcgtcactaccgctttggcgcttccagcctgctggcaagcctgctgaaagcgctgggtcacggcgacggtaagagcgccagcagctactaa

SEQ ID NO: 34 - DNA, 42 bp, Artificial Sequence
  Synthesized
cgcgagcgaaagcggcggtatggaactgcttttattgagtaa

SEQ ID NO: 35 - DNA, 29 bp, Artificial Sequence
  Synthesized
aagctggtcaccgtttttaactcgagcgg

SEQ ID NO: 36 - DNA, 30 bp, Artificial Sequence
  Synthesized
catgccatggcaaaattagagactgttact

SEQ ID NO: 37 - DNA, 36 bp, Artificial Sequence
  Synthesized
cgctttcgctcgcgccaccatacgctgggttcagct

SEQ ID NO: 38 - DNA, 29 bp, Artificial Sequence
  Synthesized
catgccatggaactgcttttattgagtaa

SEQ ID NO: 39 - DNA, 54 bp, Artificial Sequence
  Synthesized
catgccatggcacatcaccaccaccatcacatggaactgcttttattgagtaac

SEQ ID NO: 40 - DNA, 24 bp, Artificial Sequence
  Synthesized
catgccatggaagacgccaaaaac

SEQ ID NO: 41 - DNA, 29 bp, Artificial Sequence
  Synthesized
gcggaaagtccaaattgtaactcgagcgg

SEQ ID NO: 42 - DNA, 33 bp, Artificial Sequence
  Synthesized
ctattctgattacacccaaaggggatgataaac

SEQ ID NO: 43 - DNA, 33 bp, Artificial Sequence
  Synthesized
gtttatcatcccctttgggtgtaatcagaatag

(19) United States
(12) Patent Application Publication
(10) Pub. No.: US 2013/0224828 A1
(43) Pub. Date: Aug. 29, 2013

(54) RNA-Directed Packaging of Enzymes Within Protein Particles

(71) Applicants: M.G. Finn, San Diego, CA (US); Steven Brown, San Diego, CA (US); Jason Fiedler, Bonita, CA (US)

(72) Inventors: M.G. Finn, San Diego, CA (US); Steven Brown, San Diego, CA (US); Jason Fiedler, Bonita, CA (US)

(73) Assignee: QAPSULE TECHNOLOGIES, INC., San Diego, CA (US)

(21) Appl. No.: 13/882,773

(22) PCT Filed: Nov. 1, 2011
(86) PCT No.: PCT/US11/58859

§ 371 Date: May 1, 2013

Related U.S. Application Data
(60) Provisional application No. 61/408,843, filed on Nov. 1, 2010.

(51) Int. Cl. C12N 9/96

(52) U.S. Cl. CPC C12N 9/96
USPC 435/188; 435/252.3; 435/252.33; 435/254.2; 435/257.2; 435/325; 435/348

(57) ABSTRACT

Protein nanoparticles encapsulate cargo proteins within an enclosure containing a protected chemical milieu. Encapsulation within such protected chemical milieu enhances the employability and performance of cargo proteins, particularly cargo enzymes, particularly within otherwise hostile chemical environments. Protein nanoparticles are assembled using shell proteins, such as viral coat proteins like Qβ, in the presence of a bifunctional polynucleotide and the selected cargo protein. The bifunctional polynucleotide includes two aptameric activities that assist the disposition and retention of cargo proteins within the protein nanoparticle.

### STATEMENT OF GOVERNMENT SUPPORT

This invention was made with government support under grant number RR021886, awarded by the National Institutes of Health. The U.S. government has certain rights in the invention.

### FIELD OF INVENTION

The invention relates to enzymology. More particularly, the invention relates to encapsulated enzymes.

### BACKGROUND

The sequestration of functional units from the environment is a hallmark of biological organization. In addition to encapsulation within lipid membrane-bound organelles, proteinaceous cages serve this purpose for many prokaryotes. See, for example, C. A. Kerfeld, et al., Science 2005, 309, 936. From a chemical perspective, the outstanding advantages of such packages are their capabilities for high selectivity and activity, both achieved by encapsulating only those catalysts required for the desired task in a confined space, and the potential for the container to control its position in a complex environment. Artificial encapsulation or immobilization on solid supports has been shown to confer stability as well as facilitate purification and reuse. See, for example, W. Tischer, et al., Top. Curr. Chem. 1999, 2000, 95; and U. Hanefeld, et al., Chem. Soc. Rev. 2009, 38, 453. While chemists have sequestered enzymes in or on a wide variety of non-biological compartments, Nature remains the undisputed master of the art.

Protein nano-particles represent a uniquely useful bridge between chemistry, materials science, and biology because they combine robust self-assembly properties with genetically-enabled atomic control of chemical reactivity. The synthetic biomimetic packaging of functional proteins has been accomplished with several different types of protein nano-particles. Two general strategies have been employed. Synthetic biomimetic packaging of functional proteins has been achieved by genetic fusion of the cargo to a component that directs localization to the particle interior. See, for example, G. Beterams, et al., FEBS Letters 2000, 481, 169; V. A. Kickhoefer, et al., Proc. Nat'l. Acad. Sci. U.S.A. 2005, 102, 4348; F. P. Seebeck, et al., J. Am. Chem. Soc. 2006, 128, 4516; T. Inoue, et al., J. Biotechnol. 2008, 134, 181; L. E. Goldsmith, et al., ACS Nano 2009, 3, 3175; and I. J. Minten, et al., J. Am. Chem. Soc. 2009, 131, 17771. Alternatively, synthetic biomimetic packaging of functional proteins has also been achieved by non-specific packaging by in vitro assembly. See, for example, K. W. Lee, et al., J. Virol. Methods 2008, 151, 172; and M. Comellas-Aragones, et al., Nat. Nanotech. 2007, 2, 635. Early work in this field was performed by P. G. Stockley and co-workers who described the potential of engineered modular packaging in MS2 particles. See, for example, M. Wu, et al., Bioconjugate Chem. 1995, 6, 587-595; and W. L. Brown, et al., Intervirology 2002, 45, 371-380. However, yields of the encapsulated protein products have been low, and, while examples of increased stability towards a variety of treatments have been noted, no quantitative kinetic comparisons of enzymes in free vs. protein-encapsulated forms have been described.

Bacteriophage Qβ is known to form icosahedral protein nanoparticles from 180 copies of a 14.3 kD coat protein (CP). See, for example, T. M. Kozlovska, et al., Gene 1993, 137, 133; and R. Golmohammadi, et al., Structure 1996, 4, 543. These nanoparticles have been shown to be highly stable under a variety of conditions and have been used to display functional small molecules on their exterior surface. See, for example, E. Strable and M. G. Finn, Curr. Top. Microbial. Immunol. 2009, 327, 1. These nanoparticles have also been shown to display immunogenic ligands on their exterior surface. See, for example, E. Kaltgrad, et al., ChemBioChem 2007, 8, 1455; and J. Comuz, et al., PLoS ONE 2008, 3, e2547. And finally, these nanoparticles have also been shown to display peptides and proteins on their exterior surface. See, for example, I. Vasiljeva, et al., FEBS Letters 1998, 431, 7; and D. Baneijee, et al., ChemBioChem 2010. The infectious phage particle is known to package its single-stranded RNA genome by virtue of a high-affinity interaction between a hairpin stricture and interior-facing residues of the coat protein. See, for example, G. W. Witherell and O. C. Uhlenbeck, Biochemistry 1989, 28, 71. This interaction is preserved when the coat protein is expressed recombinantly to form nanoparticles. See, for example, H. Weber, Biochim. Bioophys. Acta 1976, 418, 175.

What was needed was a general, robust, and modular methodology for encapsulating recombinant enzymes and other cargo proteins within the interior space of protein nanoparticles.

### SUMMARY

One aspect of the present invention is directed to protein nano-particles that encapsulate cargo proteins within an enclosure containing a protected chemical milieu. Encapsulation within such protected chemical milieu can impart enhanced employability and performance to cargo proteins, particularly within otherwise harsh chemical environments. Protein nano-particles are assembled using shell proteins, such as viral coat proteins like Qβ, in the presence of a bifunctional polynucleotide and the selected cargo protein. The bifunctional polynucleotide includes two aptameric activities that assist the disposal and retention of cargo proteins within the protein nano-particles.

One aspect of the invention is directed to a synthetic capsule construct for providing a protected chemical milieu. The construct comprises a shell, a cargo protein, and a bifunctional polypeptide. The shell has a plurality of shell proteins; the plurality of shell proteins are assembled with one another for forming the shell and defining an enclosure therein. Each of the shell proteins, when assembled for forming the shell, has an interior surface facing inwardly toward the enclosure and an exterior surface facing outwardly away from the enclosure. The shell serves to restrict permeability to and from the enclosure and provides the protected chemical milieu therein. The shell proteins are recombinant. The cargo protein is recombinant and optionally includes a peptide tag. The bifunctional polynucleotide has both a first aptameric activity for binding the cargo protein and a second aptameric activity for retaining the bifunctional polynucleotide within the enclosure by assembly with the interior surface of the shell protein. The bifunctional polynucleotide is non-naturally occurring; the bifunctional polynucleotide serves to link the cargo protein within the enclosure for providing the cargo protein with the protected chemical milieu therein. In a preferred embodiment the cargo protein includes a tag. Another preferred embodiment selects the peptide tag from a group consisting of a peptide sequence genetically grafted onto the cargo protein and a peptide sequence evolved within the cargo protein. In another preferred embodiment, the first aptameric activity is grafted into the bifunctional polynucleotide as an aptamer evolved for binding activity with respect to the tag. Another preferred embodiment selects the cargo protein from a group consisting of enzymes and signaling proteins; more particularly, the cargo protein may be selected from a group consisting of peptide cleavage enzymes, ester cleavage and formation enzymes, phosphate cleavage and formation enzymes, glycosyl transferases, alkylating enzymes, oxidases, reductases, dehydrating and hydrating enzymes, decarboxylases, carboxylases, aldolases, transaldolases, carbon-nitrogen lyases, nitrogen transferases, ammonia lyases, pyridoxal-requiring enzymes, isomerizing enzymes, prenyl group transferases, rearrangement enzymes, acetate, and priopionate fatty acid synthases. Another preferred embodiment selects the shell protein from a group consisting of capsid proteins, coat proteins, and envelope proteins. In particular, the shell protein may be Qβ capsid protein; alternatively, the shell protein may be of a type derived from a single-stranded RNA virus, for example, icosahedral virus, bromovirus, comoviruses, nodavirus, picornavirus, tombusviruses, levivirus, or tymovirus. In another preferred embodiment, the shell protein is of a type derived from a double-stranded RNA virus, for example, birnavirus and reovirus. In another preferred embodiment, the shell protein is of a type derived from a double-stranded DNA virus, for example, parvovirus, microvirus, podovirus, or polyomavirus. In another preferred embodiment, the shell protein is a non-viral recombinant protein capable of self-assembly to form a synthetic capsule construct, for example, lumazine synthase, ferritin, carboxysome, encapsulin, vault protein, GroEL, or heat shock protein. In another preferred embodiment, the interior surface of the shell protein includes an inner surface receptor site against which the second aptameric activity includes binding activity. In another preferred embodiment, the bifunctional polynucleotide is RNA, for example, transcribed RNA from plasmid pET. In particular, the second aptameric activity of the bifunctional polynucleotide may have a binding activity with respect to an inner surface receptor site on the shell protein; alternatively, the second aptameric activity of the bifunctional polynucleotide may have non-specific binding affinity for the inner surface of the shell protein. In another preferred embodiment, the bifunctional polynucleotide is DNA. In particular, the second aptameric activity of the bifunctional polynucleotide may have a binding activity with respect to an inner surface receptor site on the shell protein; alternatively, the second aptameric activity of the bifunctional polynucleotide may have non-specific binding affinity for the inner surface of the shell protein. In another preferred embodiment, the synthetic capsule construct is capable of binding to a target and further comprises an address ligand conjugated to the exterior surface of the shell protein for binding the construct to the target.

Another aspect of the invention is directed to a synthetic tri-molecular construct comprising a shell protein, a cargo protein and a bifunctional polynucleotide. Both the shell protein and cargo protein are recombinant. The cargo protein optionally includes a peptide tag. The bifunctional polynucleotide has both a first aptameric activity for binding the cargo protein and a second aptameric activity for binding the shell protein. The bifunctional polynucleotide is non-naturally occurring; the bifunctional polynucleotide is linked both to the cargo protein and to the shell protein. In an alternative embodiment, the cargo protein includes the tag.

Another aspect of the invention is directed to a synthetic bi-molecular shell construct capable of binding a cargo protein. The construct comprises a shell protein and a bifunctional polynucleotide. The shell protein is recombinant. The bifunctional polynucleotide has both a first aptameric activity for binding the cargo protein and a second aptameric activity for binding the shell protein. The bifunctional polynucleotide is non-naturally occurring; the bifunctional polynucleotide is linked to the shell protein and is capable of linkage to the cargo protein.

Another aspect of the invention is directed to a synthetic bi-molecular cargo construct capable of binding a shell protein. The construct comprises a cargo protein and a bifunctional polynucleotide. The cargo protein is recombinant and optionally includes a peptide tag. The bifunctional polynucleotide has both a first aptameric activity for binding the cargo protein and a second aptameric activity for binding the shell protein. The bifunctional polynucleotide is non-naturally occurring; the bifunctional polynucleotide is linked to the cargo protein and is capable of linkage to the shell protein.

Another aspect of the invention is directed to a process for assembling a synthetic capsule construct. The process comprises the steps of combining and linking. In the combining step, a plurality of shell proteins are combined together with one or more cargo proteins in the presence of one or more bifunctional polynucleotides under conditions for assembling the synthetic capsule construct. The shell proteins are assembled with one another to form a shell and define an enclosure therein. Each of the shell proteins, when assembled for forming the shell, has an interior surface facing inwardly toward the enclosure and an exterior surface facing outwardly away from the enclosure. The shell proteins are recombinant; the cargo proteins are also recombinant and optionally include a peptide tag. The bifunctional polynucleotide has both a first aptameric activity for binding the cargo protein and a second aptameric activity for retaining the bifunctional polynucleotide within the enclosure by assembly with the interior surface of the shell protein. The bifunctional polynucleotide is non-naturally occurring. In the linking step, the bifunctional polynucleotide is linked to the cargo protein for retaining the cargo protein within the enclosure of the synthetic capsule construct. An alternative mode of the process selects the cargo protein from a group consisting of peptide cleavage enzymes, ester cleavage and formation enzymes, phosphate cleavage and formation enzymes, glycosyl transferases, alkylating enzymes, oxidases, reductases, dehydrating and hydrating enzymes, decarboxylases, carboxylases, aldolases, transaldolases, carbon-nitrogen lyases, nitrogen transferases, ammonia lyases, pyridoxal-requiring enzymes, isomerizing enzymes, prenyl group transferases, rearrangement enzymes, acetate, and priopionate fatty acid synthases. In another alternative mode, the combination and linking steps occur within a host cell containing one or more plasmids encoding the shell proteins, the cargo proteins, and the bifunctional polynucleotides. In another alternative mode, the combination step occurs extra-cellularly under in vitro conditions. In another alternative mode, a further step conjugates an address ligand to the exterior surface of one or more of the shell proteins.

Another aspect of the invention is directed to a process for protecting a cargo protein from a solute. The process comprises the steps of confining and exposing. In the confining step, a cargo protein is confined within the enclosure of a synthetic capsule construct by linkage with a bifunctional polynucleotide. The synthetic capsule construct is of a type affording protection from the solute. Then, in the exposing step, the synthetic capsule construct is exposed to the solute, whereby the cargo protein is protected from the solute by enclosure within the synthetic capsule construct. In an alternative mode, there are further steps of conjugating and binding. In the conjugating step, an address ligand is conjugated to the synthetic capsule construct. The address ligand has binding activity with respect to a target having an adhesion activity with respect to the address ligand. Then, in the binding step, the synthetic capsule construct is bound to a target by adhesion to the address ligand, whereby the cargo protein becomes located adjacent to the target adhesion to the address ligand conjugated to the synthetic capsule construct. A further alternative mode selects the cargo protein from a group consisting of peptide cleavage enzymes, ester cleavage and formation enzymes, phosphate cleavage and formation enzymes, glycosyl transferases, alkylating enzymes, oxidases, reductases, dehydrating and hydrating enzymes, decarboxylases, carboxylases, aldolases, transaldolases, carbon-nitrogen lyases, nitrogen transferases, ammonia lyases, pyridoxal-requiring enzymes, isomerizing enzymes, prenyl group transferases, rearrangement enzymes, acetate, and priopionate fatty acid synthases.

Another aspect of the invention is directed to a host cell for producing a synthetic capsule construct. The host cell comprises a first polynucleotide expressible for producing a recombinant cargo protein, a second polynucleotide expressible for producing a recombinant shell protein, and a third polynucleotide transcribable for producing a bifunctional polynucleotide. The bifunctional polynucleotide is capable of linking the recombinant shell proteins to the recombinant cargo proteins for assembly into a synthetic capsule construct. The first, second, and third polynucleotides are embedded in one or more potentially overlapping polynucleotides selected from a group consisting of plasmid polynucleotides and genomic polynucleotides. In an alternative to this mode of the invention, the cargo protein is selected from a group consisting of peptide cleavage enzymes, ester cleavage and formation enzymes, phosphate cleavage and formation enzymes, glycosyl transferases, alkylating enzymes, oxidases, reductases, dehydrating and hydrating enzymes, decarboxylases, carboxylases, aldolases, transaldolases, carbon-nitrogen lyases, nitrogen transferases, ammonia lyases, pyridoxal-requiring enzymes, isomerizing enzymes, prenyl group transferases, rearrangement enzymes, acetate, and priopionate fatty acid synthases.

Cargo proteins can be the native form, or can be modified with a binding domain, or “tag”, adapted to bind to an aptamer sequence. The enzymes are sequestered within the protein nano-particle through a strong association with the interior of the protein nano-particle wall through a bifunctional polynucleotide linker. In a preferred mode, the bifunctional polynucleotide linker includes a binding sequence for the complementary binding domain of the enzyme (which can be a domain of the native enzyme, or can be an engineered tag sequence of an enzyme-tag hybrid polypeptide), and another sequence that binds to sites on the protein nano-particle interior wall. The entire structure of capsid-bifunctional polynucleotide linker-cargo enzyme can self-assemble after biological production in an organism such as E. coli or yeast. The enzymes thus nanoencapsidated can be found to be active and more stable in a number of ways than analogous enzymes free in solution; for example, the encapsidated enzymes are less subject to both proteolytic and thermal degradation/denaturation than are the free enzymes. The catalytic nano-particles can be used in a variety of applications.

In various embodiments, the invention provides a catalytic nano-particle comprising a protein nano-particle with one or more types of encapsidated cargo enzyme, the protein nano-particle comprising a self-assembled capsid structure comprising multiple copies of a capsid protein, within which capsid structure is disposed one or more copies of each of the one or more types of cargo enzyme, each type of cargo enzyme being associated by a binding interaction with a respective aptamer incorporated into a bifunctional polynucleotide copy, each polynucleotide copy comprising both a sequence for binding at least one of the cargo enzymes, and a sequence for binding to a respective interior-facing domain of the capsid protein disposed on the self-assembled capsid interior wall. In various embodiments, a cargo enzyme can be a hybrid polypeptide incorporating the enzyme sequence and an engineered tag sequence that is adapted to bind to a known aptamer domain. In other embodiments, the cargo enzyme can be a native enzyme, wherein an aptamer domain can bind to a domain of the native enzyme, provided that binding does not interfere with the enzyme's catalytic site. One or more copies of one or more types of cargo enzyme can be encapsidated within a single protein nano-particle, wherein each type of cargo enzyme can either bear an engineered tag for binding to the aptamer domain, or can bind to the aptamer domain via a native domain of the enzyme.

For example, in various embodiments, the invention provides a catalytic nano-particle comprising a protein nano-particle with encapsidated cargo enzyme, the protein nano-particle comprising a self-assembled capsid structure comprising multiple copies of a capsid protein, such as a Qβ capsid protein, within which capsid structure is disposed one or more copies of a tagged cargo enzyme, such as a Rev-tagged cargo enzyme, each tagged cargo enzyme being associated by a binding interaction with a respective polynucleotide copy, each polynucleotide copy comprising both an aptamer sequence, such as a Rev aptamer sequence, for binding the tagged enzyme, and a sequence, which can be a Qβ hairpin sequence, for binding to a respective interior-facing domain of the capsid protein disposed on the self-assembled capsid interior wall.

In other embodiments, the tag sequence on the cargo enzyme can be the SelB protein or a domain thereof, and the bifunctional polynucleotide can include an aptamer that binds to the SelB domain.

In various embodiments, the bifunctional polynucleotide sequence for binding to the tagged cargo protein or the interior-facing domain of the capsid protein can be a sequence that can be modified to bind with varying affinities, so as to allow changing the number of cargo proteins encapsidated within a single protein nano-particle capsid shell.

In other embodiments, the cargo enzyme can be any enzyme that can be expressed in E. coli or in yeast, and that can fit inside the capsid. Cargo enzymes can be composed of multimeric units. In various embodiments, a cargo enzyme can be a hydrolase such as a peptidase, lipase, esterase, or phosphatase, a deaminase such as cytosine deaminase, a superoxide dismutase, a mono-oxygenase such as luciferase, or a phosphorylase such as purine-deoxynucleoside phosphorylase or uracil phosphoribosyltransferase. An enzyme can be selected from among the repertoire of known enzymes based upon the catalytic activity the selected enzyme is known to possess, to carry out a desired chemical reaction using the catalytic nano-particles of the invention.

In various embodiments, a cargo enzyme thus encapsidated can have greater stability, for example, during heat, proteolysis, and absorption, than the same enzyme free in solution under comparable conditions. Thus, encapsidation as disclosed herein can serve to stabilize an enzyme under extreme conditions.

In various embodiments, the invention provides a method of preparing the catalytic nano-particle of the invention, comprising in vivo expression of vectors, the vectors together coding for all the capsid protein, the cargo enzyme, optionally including a tag sequence, and the bifunctional polynucleotide, comprising a cargo enzyme binding sequence and interior capsid wall-binding sequence, in a suitable expression system.

In various embodiments, the invention provides a first plasmid comprising capsid protein RNA, α-Rev aptamer disposed upstream of the ribosome binding site, and the Qβ hairpin disposed immediately downstream of the stop codon. In various embodiments, the invention provides a second plasmid comprising a coding sequence for a cargo enzyme N-terminally tagged with the peptide sequence, such as a Rev peptide sequence. In various embodiments, both plasmids can be used in a suitable expression system, such as E. coli or yeast, to provide the self-assembled protein nano-particle containing the bound cargo enzyme. In various embodiments, the capsid protein gene can be integrated into the chromosome of the host organism, such as E. coli or yeast.

In various embodiments, the invention provides a method of catalyzing a reaction in solution, comprising contacting a reaction-starting material with the catalytic nano-particle of the invention, or a catalytic nano-particle prepared by the method of the invention, in solution, under conditions suitable for the reaction to occur.

In various embodiments, the invention provides a nanostructured construct comprising a plurality of nano-particles of the invention or of nano-particles prepared by the method of the invention, disposed within a structured matrix. The nanostructured construct can be used to catalyze a chemical reaction, by dispersing the construct in a solution comprising the reaction-starting material(s) or passing such as solution through a porous embodiment of the nanostructured construct.

### BRIEF DESCRIPTION OF DRAWINGS

FIG. 1 illustrates a packaged molecular machine wherein peptidase E and luciferase were encapsulated and shown to be catalytically active inside the particle. Protein nano-particle assembly and encapsulation of peptidase E and luciferase were achieved using dual expression vectors that guide the preparation of Qβ virus-like particles for encapsulating multiple enzymes.

FIG. 2 illustrates a schematic representation of the technique used to package protein inside Qβ protein nano-particles.

FIG. 3 illustrates an enlarged detail of the tri-molecular construct comprising a protein shell and a cargo protein linked by a bifunctional polynucleotide.

FIG. 4 illustrates the physical characterization of Qβ@(RevPepE)18 by means of: (A) electrophoretic analysis; (B) transmission electron micrography; and (C) size-exclusion FPLC.

FIGS. 5 (A) and (B) illustrate the kinetics of PepE-catalyzed hydrolysis of fluorogenic Asp-AMC as a function of encapsidation.

FIGS. 6 (A) and (B) illustrate the protection afforded by encapsidation of peptidase E with respect to thermal and protease inactivation. Two graphs illustrate the relative activity of encapsidated and unencapsidated enzyme as a function of incubation temperature or incubation time, respectively.

FIG. 7 illustrates a scheme showing two general paths for producing protein nano-particles, viz. cellular and cell-free, in vitro.

FIG. 8 illustrates a scheme for the derivatization of Qβ@GFP 15 with glycan ligands LacNAc (using 1) and the BPC derivative of sialic acid (using 2) by Cu-catalyzed azide-alkyne cycloaddition chemistry.

### DETAILED DESCRIPTION

The invention is directed to a biologically produced, self-assembling catalytic nano-particle, comprising a hollow, porous, protein nano-particle containing one or more copies of an encapsidated cargo enzyme bound to the nano-particle interior wall; to methods of making the catalytic nano-particle, to methods of using the catalytic nano-particle, and to a nanostructured construct comprising a plurality of catalytic nano-particles of the invention disposed within a matrix.

In various embodiments, the protein nano-particle comprises a self-assembled capsid or capsid-like structure comprising multiple copies of a protein, such as a capsid protein, within which structure is disposed one or more copies of one or more types of a cargo enzyme, each cargo enzyme being associated by a binding interaction with a respective RNA copy, each RNA copy comprising (a) a sequence for binding the cargo enzyme, and (b) a sequence for binding to a respective interior-facing domain of the capsid protein disposed on the self-assembled capsid interior wall. In various embodiments, the enzyme can be a hybrid polypeptide incorporating the enzyme sequence and an engineered tag sequence that is adapted to bind to an RNA aptamer domain. For example, the engineered enzyme tag sequence can be a Rev peptide sequence, adapted to bind a Rev aptamer RNA sequence. In other embodiments, the RNA domain can bind to a domain of a native enzyme, provided that the binding does not interfere with the enzyme's catalytic site.

One or more copies of one or more types of cargo enzyme can be encapsidated within a single protein nano-particle, wherein each type of cargo enzyme can bear an engineered tag for binding to the RNA, or can bind to the RNA via a native domain of the enzyme. In various embodiments, each nano-particle comprises a single type of enzyme. In other embodiments, each nano-particle can comprise multiple types of cargo enzymes, such as a set of enzymes that together carries out a complex metabolic or synthetic transformation or transformations.

For example, the capsid protein can be a Qβ capsid protein, which self-assembles to form a protein nano-particle containing 180 copies of the 14.3 kD coat (capsid) protein. Other self-assembling capsid proteins can be used, provided they have binding sites for an RNA sequence disposed on the interior wall of the capsid.

For example, the tagged cargo enzyme can be a Rev-tagged cargo enzyme, wherein the Rev sequence is an arginine-rich peptide derived from HIV-1. The Rev sequence can be disposed N-terminally to the enzymic peptide sequence, or can be disposed C-terminally.

For example, the RNA sequence for binding the tagged enzyme can be a Rev sequence, an aptameric sequence developed by in vitro selection to bind the Rev peptide sequence.

For example, the RNA sequence for binding to the interior facing domain of the capsid protein can be a Qβ hairpin sequence. Alternatively, another RNA sequence adapted to bind a protein domain of another self-assembling capsid protein can be used.

In various embodiments, the invention provides a bifunctional RNA sequence comprising an enzyme binding sequence, such as a tagged enzyme binding sequence, and a capsid interior wall domain binding sequence, that respectively bind a cargo enzyme, such as by tag peptide sequence, and a capsid protein interior wall binding domain. This construct serves to effectively immobilize or bind the cargo enzyme to the capsid interior, thus forming the catalytic nano-particle of the invention.

The cargo enzyme, i.e., the enzyme to be sequestered within the capsid shell, can be any suitable enzyme(s) which can be produced biologically, either with the selected tagging peptide sequence, such as the Rev peptide sequence, or wherein a domain of the native enzyme binds the bifunctional RNA within the capsid structure. The enzyme and tagging sequence hybrid can be produced using biological techniques known in the art, such as through in vivo expression of an engineered plasmid.

In various embodiments, the cargo enzyme can be a can be a hydrolase such as a peptidase, lipase, esterase, or phosphatase, a deaminase such as cytosine deaminase, a superoxide dismutase, a monooxygenase such as luciferase, or a phosphorylase such as purinedeoxynucleoside phosphorylase or uracil phosphoribosyltransferase. For example, the cargo enzyme can be a peptidase or a luciferase, as is exemplified below. Each of the one or more types of cargo enzyme can be any desired enzyme that can function in a cytosolic environment, including cytosolic domains of membrane-spanning enzymes or receptors. It is only necessary that the enzyme be capable of expression in organisms such as E. coli or yeast, and that they spatially fit within the capsid.

For example, a cargo enzyme can be a functional domain of a larger natural protein, wherein the functional domain has a desired catalytic activity. A cargo enzyme can thus be an engineered enzyme that may or may not have a natural counterpart, provided that the enzyme can be encoded in a gene that can be expressed in organisms such as E. coli or yeast.

The catalytic nano-particle of the invention can include a cargo enzyme that is adapted to catalyze a reaction wherein the reaction starting material is a cargo enzyme substrate, such that when the catalytic nano-particle and the reaction starting material are contacted in solution, the cargo enzyme catalyzes the reaction, optionally wherein the solution is a substantially aqueous solution. An enzyme can be selected that catalyzes a reaction of interest in solution, and a catalytic nano-particle of the invention can be prepared using the methods of the invention, wherein the catalytic nano-particle serves to catalyze the desired reaction. The encapsidation of the enzyme, i.e., the cargo enzyme, within the capsid, both allows for easier chemical processing of the reaction, and confers enhanced stability on the enzyme, under a variety of conditions.

In various embodiments, the coat (capsid) protein can embody various properties, such as differential stability to heat, pH, salt, or reducing conditions; or differential vasculature lifetimes or affinity to cell types. An engineered coat protein sequence can include a functional domain, that can serve to form hybrid particles without changing the packaging properties. The functional domain can be a small peptide or entire protein. Further, the number of engineered domains can be modulated by changing expression conditions. The functional domain can facilitate entry into cell, escape from interior organelles, trafficking within the cell, immune evasion or immune amplification. See, for example, S. D. Brown, et al., “Assembly of hybrid bacteriophage Qβ virus-like particles”, Biochemistry 2009, 48 (47), 11155-11157.

It has been found by the inventors herein that encapsidation of an enzyme in a catalytic nano-particle of the invention provides enhanced stability for the enzyme under a variety of conditions, compared to the stability of the same enzyme in solution under comparable conditions.

In various embodiments, the invention provides a catalytic nano-particle wherein the cargo enzyme disposed within the protein nano-particle capsid is more resistant to degradation or denaturation than is the cargo enzyme free in solution under comparable conditions, optionally wherein the solution is a substantially aqueous solution.

For example, the invention provides a catalytic nano-particle wherein the cargo enzyme disposed within the protein nano-particle dispersed in solution is more resistant to thermal degradation or denaturation than is the cargo enzyme free in solution at the same temperature, optionally wherein the solution is a substantially aqueous solution.

For example, the invention provides a catalytic nano-particle wherein the cargo enzyme disposed within the protein nano-particle dispersed in solution is more resistant to degradation by a dissolved proteolytic enzyme than is the cargo enzyme free in solution in the presence of a comparable concentration of the proteolytic enzyme, optionally wherein the solution is a substantially aqueous solution.

For example, the invention provides a catalytic nano-particle wherein the cargo enzyme disposed within the protein nano-particle dispersed in solution is more resistant to loss of catalytic activity by absorption onto a surface than is the cargo enzyme free in solution in the presence of a comparable surface, optionally wherein the solution is a substantially aqueous solution.

In various embodiments, the catalytic nano-particle can be engineered to encapsidate various copy numbers of the cargo enzyme. For example, in various embodiments, each nano-particle can comprise between 1 and 50 copies of the cargo enzyme; or each nano-particle can comprise between 2 and 18 copies of the cargo enzyme.

In various embodiments, the invention provides a method of preparing the catalytic nano-particle of the invention, the method comprising in vivo expression of expression vectors, the vectors together coding for all of: the capsid protein, the tagged cargo enzyme, and the bifunctional RNA comprising a tagged cargo enzyme binding sequence and interior capsid wall binding sequence, in a suitable expression system.

For example, the capsid protein can be a Qβ capsid protein, which can be produced by expression of the Qβ coding sequence engineered in a plasmid in an organism, such as the E. coli organism, using suitable promoters and the like, as is well known in the art. The Qβ capsid protein self-assembles into the protein nano-particle upon expression.

For example, tagged cargo enzyme can be a Rev-tagged cargo enzyme, produced in a similar manner from a suitably engineered plasmid in an organism such as E. coli, the plasmid containing a coding sequence along with the arginine-rich Rev tag sequence, using suitable promoters and the like, as is well known in the art. For example, the tagged cargo enzyme binding sequence of the bifunctional RNA can comprise a Rev-binding sequence, an interior capsid wall-binding sequence comprising a Qβ hairpin (hp) sequence, or both. The expression system can be any suitable living organism.

In various embodiments, the invention provides a first plasmid coding for the capsid protein and the bifunctional RNA, and a second plasmid coding for the tagged cargo enzyme.

For example, the first plasmid can code for a Qβ capsid protein and an RNA containing a Qβ hairpin binding sequence; the second plasmid can code for a Rev-tagged cargo enzyme. The plasmids can contain suitable promoters, stop codons, and the like, as are well known in the art.

As discussed above, the plasmid coding the cargo enzyme plus peptide tag sequence can be engineered as is well known in the art to include coding sequences for any suitable enzyme of known sequence, combined with any suitable peptide tagging sequence. The cargo enzyme can be selected to catalyze a suitable reaction of interest, for example a hydrolytic reaction, such a cleavage of a peptide or a phosphate group. Accordingly, the plasmid can be engineered to include a coding sequence for the peptide tag sequence in conjunction with a hydrolase or a phosphatase, such as a peptidase or a luciferase, respectively.

In various embodiments, and as described in greater detail below, a first plasmid can comprise capsid protein RNA, a Rev aptamer disposed upstream of the ribosome binding site and the Qβ hairpin disposed immediately downstream of the stop codon. More specifically, the first plasmid can be a ColE1-group plasmid.

In various embodiments, and as described in greater detail below, a second plasmid can comprise a coding sequence for a cargo enzyme N-terminally tagged with the Rev peptide sequence. More specifically, the second plasmid can be a compatible CloDF13-group plasmid. In various embodiments, the method of the invention can comprise expressing both plasmids in a living organism, such as E. coli or yeast, containing both plasmids.

In various embodiments, the invention provides a method of catalyzing a reaction in solution, comprising contacting a reaction-starting material with the catalytic nano-particle of invention or a catalytic nano-particle prepared by the method of invention, in solution, under conditions suitable for the reaction to occur. For example, the solution can be a substantially aqueous solution, and the reaction can be a hydrolytic reaction, or both. The catalytic nano-particle can be engineered without undue experimentation to contain any suitable enzyme for carrying out a reaction of interest, provided an enzyme can be identified, and its coding sequence determined, that is suitable for catalyzing the reaction of interest.

The solvent need not be limited to a purely aqueous solvent. In various embodiments, the reaction solution can comprise an organic solvent or solvents, with or without water. This can provide for the catalysis of reactions involving poorly water-soluble substrates, e.g., lipids, using the catalytic nano-particle. Suitable organic solvents that are miscible or at least soluble in water include lower alcohols (ethanol), lower amides (DMF, NMP), DMSO, glycols, and the like, as discussed further below.

In various embodiments, the invention provides a nanostructured construct comprising a plurality of nano-particles of the invention or of nano-particles prepared by the method of the invention, disposed within a structured matrix. Such materials can be used as catalysts with favorable properties for carrying out large scale chemical transformation. For example, the construct can be dispersed in a solvent, or can be disposed on or within a porous material through which a solvent can pass, so as to catalyze a chemical reaction of a reaction substrate dissolved in the solvent, as discussed further below.

In various embodiments, the invention provides a nanostructured construct comprising a plurality of nano-particles of the invention or of nano-particles prepared by the method of the invention, administered to a patient for the purpose of therapeutic or diagnostic application. For example, a particle containing superoxide dismutase can be administered for the treatment of inflammatory bowel disease or rheumatoid arthritis, superoxide being a known inflammatory agent in these conditions.

To facilitate RNA-directed encapsidation, two binding domains were introduced to the CP RNA, carried on a ColE1-group plasmid. An RNA aptamer developed by in vitro selection to bind an arginine-rich peptide (Rev) derived from HIV-1 [W. Xu and A. D. Ellington, Proc. Nat'l. Acad. Sci. U.S.A. 1996, 93, 7475] was inserted just upstream of the ribosome binding site. The sequence of the Qβ packaging hairpin was positioned immediately downstream of the stop codon. The cargo enzyme was N-terminally tagged with the Rev peptide and inserted into a compatible CloDF13-group plasmid. Transformation with both plasmids and expression in BL21(DE3) E. coli yielded protein nano-particles encapsidating the Rev-tagged protein. Such species are designated Qβ@(protein)n, where n=the average number of proteins packaged per particle, determined by electrophoretic analysis as in FIG. 2a and Supporting Figure S1a. We report here the packaging of the 25-kD N-terminal aspartate dipeptidase peptidase E (PepE) [R. A. Lassy and C. G. Miller, J. Bacter. 2000, 182, 2536], 62-kD firefly luciferase (Luc), and a thermostable mutant of Luc (tsLuc) [P. J. White, et al., Biochem. J. 1996, 319 (Pt 2), 343] inside protein nano-particles.

The enzyme-filled protein nano-particles were indistinguishable from standard protein nano-particles by techniques that report on the exterior dimensions of the particles (transmission electron microscopy, size-exclusion chromatography, and dynamic light scattering). However, the particles exhibited different densities by analytical ultracentrifugation: non-packed Qβ nano-particles, 76S; Qβ@(RevLuc)4, 79S; and Qβ@(RevPepE)18, 86S. These values agree with variations expected in overall molecular weights calculated from estimates of the RNA and protein content of each particle.

The average number of encapsidated cargo proteins was controlled by changing expression conditions or by removing interaction elements from the plasmids. In this way, PepE incorporation could be reproducibly varied between 2 and 18 per particle. Fewer copies of Luc proteins were packaged, with less variation in the number: 4-8 copies per particle were found for most conditions, whereas the number of packaged tsLuc molecules was varied between 2 and 11 per protein nano-particle. In addition to its larger size, Luc is less stable than PepE and its gene was not optimized for expression in E. coli, all factors that could contribute to the lower numbers of packaged enzyme in this case. Yields of purified particles ranged from 50-75 mg per liter of culture for the typical particles encapsidating PepE, and 75-140 mg per liter for the Luc or tsLuc particles. To test the functional capabilities of the packaged enzymes, the activities of encapsidated Rev-PepE and free PepE were compared using the fluorogenic substrate Asp-AMC (R. A. Larsen, et al., J. Bacteria 2001, 183, 3089; and I. T. Mononen, et al., Anal. Biochem. 1993, 208, 372). The kinetic parameters, obtained by standard Michaelis-Menten analysis, were found to be quite similar for the two forms of the enzyme, with k_{cat}/Kₘ for free PepE exceeding that of Qβ@(RevPepE)₉ by a factor of only three (1.8±0.2×10−2 vs. 6.3±0.9×10−3). The observed Km values are comparable to those reported for cleavage of Asp-Leu (0.3 mM) (A. Lassy and C. G. Miller, J. Bacter. 2000, 182, 2536). For this analysis, all copies of encapsidated RevPepE in Qβ@(RevPepE) were assumed to be independently and equivalently active, and the substrate and product were assumed to diffuse freely in and out of the capsid. The close correspondence between the reactions of free and encapsidated enzymes appear to support these assumptions.

Peptidase E was also significantly stabilized by encapsidation. Free PepE retained only half of its initial activity after incubation for 30 minutes at 45° C. and 20% of its activity at 50° C. (FIG. 4A). In contrast, Qβ@(RevPepE)9 showed no loss of activity at temperatures up to 50° C. for 30 minutes. Extended incubation at these temperatures showed the packaged enzyme was about 60 times more resistant than the free enzyme to thermal deactivation (Supporting Fig. S3). Heating did not disrupt the particle structure (Supporting Fig. S4), suggesting that at least partial denaturation of the packaged protein can occur inside the capsid shell. Packaged RevPepE was also protected from protease digestion, maintaining more than 80% activity under conditions in which the activity of the free enzyme was entirely degraded by proteinase K (FIG. 4B).

The activity of Qβ@(RevLuc) was similarly compared to free recombinant firefly luciferase. In this case, packaging of the enzyme did not substantially change kcat, but Km in both luciferin and ATP substrates was significantly higher for the packaged enzyme (Table 1). Luciferase is quite unstable toward thermal denaturation in both free and immobilized forms (C. Y. Wang, et al., Anal. Biochem. 1997, 246, 133), the free tsLuc variant having a half-life at 37° C. of only 16 minutes (P. J. White, et al., Biochem. J. 1996, 319 (Pt. 2), 343). No improvement in thermal sensitivity was observed for Qβ@(Rev-tsLuc)₉, but both packaged enzymes were protected from inactivation (presumably from adsorption) to unblocked polystyrene plates, to which the free enzyme was highly susceptible.

TABLE 1
Kinetic constants for free and packaged luciferase enzymes.

| | K_{m, app} | K_{m, app} | k_{cat} |
|---|---|---|---|
| | (μM), luciferin | (μM), ATP | (s⁻¹) |
| free luciferase | 7.9 ± 0.1 | 60 ± 10 | 38 ± 1.9 |
| Qβ@(RevLuc)₄ | 140 ± 7 | 460 ± 30 | 22 ± 0.4 |
| Qβ@(RevtsLuc)₂ | 77 ± 3 | 360 ± 20 | 35 ± 8 |
| Qβ@(RevtsLuc)₉ | 171 ± 8 | 550 ± 30 | 20 ± 2 |

* Calculated from specific activity of luciferase (4.89 × 10¹⁰ light units/mg) and conversion to moles of pyrophosphate released.

These results represent the first examples of polynucleotide-mediated packaging of functional enzymes inside a protein shell, and the first kinetic comparisons between free and protein-encapsulated catalysts. While some differences were noted in kinetic parameters, the free and encapsidated enzymes exhibited very similar activities at saturation on a per-enzyme basis, showing that the enzyme-filled capsids can be highly potent catalytic engines.

The RNA-mediated packaging method combines the binding functions of two linked RNA aptamers, the first a natural hairpin sequence that engages in a strong association with the inside of the protein nano-particle, and the second an artificial aptamer selected by in vitro methods to bind to an oligopeptide tag fused to the desired cargo. The fact that the second of these aptamers works is especially significant, since it shows that the active conformation of the aptamer is accessible even when the sequence is coded into a larger piece of expressed and packaged messenger RNA. The binding site on the coat protein for RNA is not a continuous feature. It has been disclosed by F. Lim, et al. (J. Biol. Chem. 1996, 271 (50), 31839-45), that approximately 10 mutations are important for RNA binding, viz., residue numbers 32, 49, 56, 59, 61, 63, 65, 89, 91, 95.

This method of packaging enzymes inside protective protein shells has several attributes that distinguish it from existing technologies. First, since the entire packaging scheme is present within the host bacteria, the complete structure is assembled by the end of the expression. There is no need to purify separate elements and bring them together in vitro as in other systems; these time-consuming steps are often low-yielding and require large amounts of starting material. Secondly, purification is largely independent of the packaged material, allowing the same efficient procedures to be used for a large range of packaged proteins. Thirdly, in contrast to most other co-expression systems [3a-e], we use a scaffold that was evolved in E. coli, and expression in the native host provides high yields of pure protein nano-particle in a short amount of time.

The active nature of the encapsulated enzymes, and the ability of the capsid shell to stabilize them against thermal degradation, protease attack, and hydrophobic adsorption, shows that this method may be generally applicable to the production of fragile or difficult-to-purify enzymes.

All production and assembly steps occur within the bacterial cell, with indirect control of amount of packaged cargo possible by simply changing the expression media or the nature of the components of the packaging system. Protein nano-particles are produced in high yields and are purified by a convenient standard procedure, independent of the protein packaged inside. This system therefore represent a unique method for the harnessing of enzymatic activity in a process-friendly fashion.

Specific Exemplifications of Packaged Enzymes

Table 2 lists enzymes that have been packaged inside Qβ virus-like particles by the above methodology. The monomeric molecular weights, number of monomers reported to be necessary to assemble into a functional enzyme, and the average number of enzyme monomers found per particle are also listed. For several examples, the catalytic activities have been measured and are provided in terms of the normal definition of Michaelis-Menten kₐₜ and Kₘ.

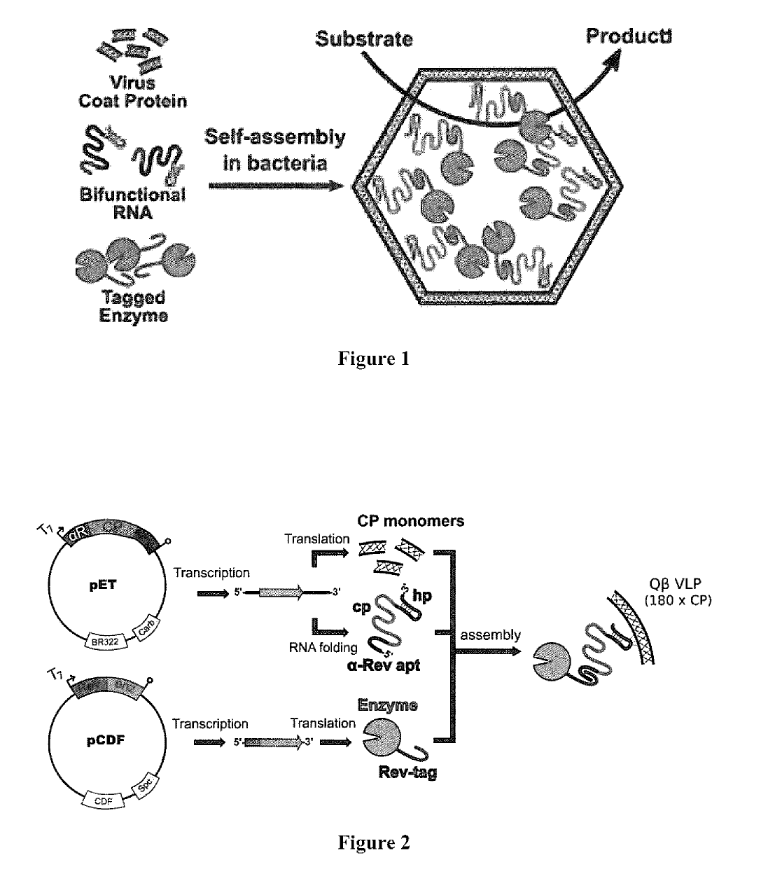

TABLE 2
Enzymes packaged inside Qβ VLPs using the Rev-peptide tag methodology shown in FIG. 1B. In most cases,
no figures appear for kinetic parameters because the assays are currently being developed or implemented.

| | | | MW | mult. | # | k_{cat} | Kₘ |
|---|---|---|---|---|---|---|---|
| Entry | Enzyme | Gene | (kDa) | state ^{a} | encapsidated ^{b} | (s⁻¹) | (μM) |
| 1 | Peptidase E | pepE | 28.1 | 1 | 2-24 | 1.7 | 270 |
| | [SEQ ID No: 4] | [SEQ ID No: 5] | | | | | |
| 2 | Luciferase | Luc | 64.2 | 1 | 4-10 | 22 | 140, 460 |
| | [SEQ ID No: 6) | [SEQ ID No: 7] | | | | | |
| 3 | Thermostable luciferase | tsLuc | 64.2 | 1 | 2-11 | 20-35 | 77 |
| | [SEQ ID No: 8] | [SEQ ID No: 9] | | | | | |
| 4 | Cytosine Deaminase | FCY1 | 21 | 2 | 16 | 4 | 470 |
| | (CD) [SEQ ID No: 10] | [SEQ ID No: 11] | | | | | |
| 5 | Cytosine Deaminase | tsFCY1 | 21 | 2 | 9-36 | 5.5-27 | 120-470 |
| | [SEQ ID No: 12] | [SEQ ID No: 13] | | | | | |
| 6 | Cytosine Deaminase | codA | 51.2 | 4 | 3-8 | 0.05 | 9300 |
| | [SEQ ID No: 14] | [SEQ ID No: 15] | | | | | |
| 7 | Uracil Phosphoribosyl- | FUR1 | | | | | |
| | transferase (UPRT) | [SEQ ID No: 17] | 28.2 | 4, 5 | 13 | | |
| | [SEQ ID No: 16] | | | | | | |
| 8 | CD-UPRT fusion | FCU1 | 45.6 | 1 | 3-6 | 6 | 400 |
| | [SEQ ID No: 18] | [SEQ ID No: 19] | | | | | |
| 9 | Purine nucleoside | PNP | 29.4 | 6 | 9-20.5 | 0.85, 42 | 70, 100 |
| | phosphorylase (PNP) | [SEQ ID No: 21] | | | | | |
| | [SEQ ID No: 20] | | | | | | |
| 10 | Asparaginase II | AnsB | 38.3 | 1 | 0.6-2.8 | | |
| | [SEQ ID No: 22] | [SEQ ID No: 23] | | | | | |
| 11 | Superoxide Dismutase A | SodA | 26.7 | 2 | 8-16 | 500-4000 U/mg ^{c} | |
| | (Mn) [SEQ ID No: 24] | [SEQ ID No: 25] | | | | | |
| 12 | Superoxide Dismutase B | SodB | 24.9 | 2 | 6.1-8.7 | | |
| | (Fe) [SEQ ID No: 26] | [SEQ ID No: 27] | | | | | |
| 13 | Superoxide Dismutase C | SodC | 19.4 | 1 | 0.7-1.1 | | |
| | (Cu/Zn) [SEQ ID No: 28] | [SEQ ID No: 29] | | | | | |
| 14 | Catalase | KatG | 83.6 | 4 | 4-8 | | |
| | [SEQ ID No: 30] | [SEQ ID No: 31] | | | | | |
| 15 | Deoxyribose-phosphate | DeoC | 31 | 1, 2 | 6.4-11 | 15 U/mg | |
| | aldolase [SEQ ID No: 32] | [SEQ ID No: 33] | | | | | |

^{a} Number of enzyme monomers involved in the catalytically-active complex.
^{b} Number of monomeric units packaged within each VLP, determined as explained above, observed during the course of “N” experiments; these numbers can vary considerably due to tests of different vectors and expression conditions.
^{c} maximum units used by commercial vendors; the free enzyme ≈ 5000 U/mg.

TABLE 3
The particles are listed that have been made containing
multiple enzymes on the inside and/or functional protein
units encoded into the virus-like particle on its outer
surface. These may be regarded as variations on the protein
shell with the same packaging method and materials inside.
Simultaneous exterior display and interior packaging
of polypeptides using Qβ VLPs.

| | | | # | # | |
|---|---|---|---|---|---|
| | packaged | displayed | pack- | dis- | |
| entry | protein * | motif | aged | played | notes |
| 1 | tsCD + PNP | None | 4 + 4 | | both enzymes |
| | | | | | independently |
| | | | | | active |
| 2 | tsCD + UPRT | None | 8 + 4 | | |
| 3 | tsCD | EGF domain | 5.6 | 5 | poor yield |
| 4 | tsCD | GE7 peptide | 4.5 | 39-50 | |
| 5 | tsCD | SDF domain | 13.6 | 23.5 | poor yield |
| 6 | tsCD | ZZ domain | 25 | 43 | new vector |
| | | | | | design |
| 7 | SOD | ZZ domain | 6.1-10.8 | 36-38 | |

* “tsCD” refers to the thermostable variant derived from the tsFCY1 gene (from S. cerevisiae).

Enzyme Types and Examples

The following list is exemplary of some of the types of enzymes that can be used as “cargo”: peptide cleavage, including proteases and peptidases; ester cleavage and formation, including esterases and lipases; phosphate cleavage and formation, including phosphatases, phosphorylases, ATPases, phosphodiesterases, kinases, and pyrophosphoryl transferases; glycosyl transferases; alkylating enzymes (typically requiring S-adenosylmethionine or tetrahydrofolate), including serine hydroxymethylase, formylases, thymidylate synthase, and methyltransferases; oxidases and reductases, including nicotinamide coenzymes, flavoprotein oxidases and dehydrogenases, hydrogenases, hydroxylases, luciferases, monooxygenases, superoxide, dismutase, hydroxylases, peroxidases, hydroperoxidases, dioxygenases, and halogenases; dehydrating and hydrating enzymes, including aconitase, fumarase, enolase, crotonase, dihydroxyacid dehydrase, dehydrases for sugar substrates (e.g., 6-phosphogluconate dehydrase), sugar biosynthetic enzymes, syn-eliminations of water: 3-methylglutaconyl-CoA dehydratase, and nitrilases; decarboxylases (α-keto acids, β-keto acids, β-hydroxy acids) and carboxylases, including acetoacetate decarboxylase, isocitrate dehydrogenase, pyruvate decarboxylase, ketol transferases, phosphoenolpyruvate (PEP) carboxylase and carboxykinase, ATP-dependent carboxylases, and ribulose-1,5-diphosphate carboxylase; aldolases and transaldolases, including standard aldolases, malate synthase, and citrate synthase; carbon-nitrogen lyases, nitrogen transferases, including ammonia lyases (e.g., aspartate-, histidine-, phenylalanine ammonia lyase); pyridoxal-requiring enzymes, including transaminases (aspartate, alanine, etc.) and racemases (alanine, arginine, etc.); isomerizing enzymes, including epimerases and racemases (e.g., proline racemase, methylmalonyl-CoA racemase, lactate racemase), aldose-ketose isomerases (e.g., triose phosphate isomerase), allylic isomerases (e.g., aconitase isomerase), phosphosugar mutases (e.g., phosphoglucomutase), and cis-trans isomerases; prenyl group transferases, including squalene synthetase, pig liver prenyl transferase, and squalene oxidocyclase; rearrangement enzymes, including alkyl migrating enzymes (e.g., acetohydroxy-acid isomeroreductase), chorismate mutase, anthranilate synthetase, and carbon rearrangements (e.g., glutamate mutase, methylmalonyl-CoA mutase); acetate and priopionate fatty acid synthases.

Peptide Tag/Aptamer Pairs Are Conventional Research Tools

Methodologies for generating new aptamers employable in a peptide tag/aptamer pair and for employing a peptide tag/aptamer pair for binding a tagged protein to an RNA strand are well known and predictable. More particularly, methodologies for generating and employing aptamers using single-stranded RNA, e.g., anti-Rev aptamer) are well developed and predictable. Large numbers of single-stranded RNA aptamers have been generated and reported. They are now considered a conventional tool for molecular biologist. See, for example, W. XU et al., Proc. Natl. Acad. Sci. USA, 93, pp. 7475-7480, July 1996; T. S. Bayer, et al., RNA (2005), 11:1848-1857; G. Hayashi, et al., J. Am. Chem. Soc. 2007, 129, 8678-8679; and R. Stoltenburg, et al., Biomolecular Engineering 24 (2007) 381-403. Methodologies for generating and employing aptamers using single-stranded DNA are also well developed and predictable. See, for example, L. C. Bock, L. C., et al., Nature 1992, 355, 564-566; C. Wang, et al., J. Biotechnol. 2003, 102, 15-22; H. Ulrich, et al., Cytometry Part A 2004, 59A, 220-231; C. S. M. Ferreira, et al., Tumor Biol. 2006, 27, 289-301; J. K. Herr, et al., Anal. Chem. 2006, 78, 2918-2924; J. A. Philips, Anal. Chem. 2006, 81, 1033-1039; D. Shangguan, et al., Proc. Natl. Acad. Sci. U.S.A. 2006, 103, 11838-11843; X. Cao, et al., Nucl. Acids Res. 2009, 37, 4621-4628; and J. Mehta, et al., J. Biotechnol. 2011, 155, 361-369.

Shell Proteins and Bifunctional Polynucleotides

Bifunctional polynucleotides are adapted to assemble/bind to the particular shell protein employed. For example, the bifunctional polynucleotide employed with Q-beta capsid protein [SEQ ID No:1 and SEQ ID No:2] incorporates an aptamer (hairpin) adapted assemble/bind to the interior face of assembled Q-beta capsid shells. The bifunctional polynucleotide [SEQ ID No:3] then serves as a linker between Q-beta capsid proteins and the encapsulated cargo proteins.

However, capsid proteins from other bacteriophages and viruses may be employed assembling synthetic capsule constructs. The bifunctional polynucleotide employed with an alternative capsid protein employs an aptamer obtained from or adapted to assemble with such virus or bacteriophage shell proteins. Bifunctional polynucleotide employable with capsids from single-stranded RNA viruses and bacteriophages are single-stranded RNA. Exemplary single-stranded RNA viruses having assemblable shell proteins employable with the present invention are as follows:

Non-icosahedral viruses (rod-shaped or other shapes): tobacco mosaic virus.
Bromoviruses: alfalfa mosaic virus, brome mosaic virus, cowpea chlorotic mottle virus, cucumber mosaic virus, tomoto aspermy virus.
Comoviruses: bean pod mottle virus, cowpea mosaic virus, tobacco ringspot virus.
Nodaviruses: black beetle virus, pariacoto virus.
Picornaviruses: coxsackievirus, echovirus, foot and mouth disease virus, rhinovirus 14, poliovirus.
Tombusviruses: artichoke mottled crinkle virus, red clover necrotic mosaic virus, tomato bushy stunt virus.
Leviviruses: bacteriophages MS2, FR, GA, PP7.
Tymoviruses: physalis mottle virus, desmodium yellow mottle virus, turnip yellow mosaic virus.

Shell proteins from virus particles that package double-stranded RNA can also be employed. However, their bifunctional polynucleotides will employ a single-stranded aptamer to bind/assemble with shell proteins. Exemplary double-stranded RNA viruses having assemblable shell proteins employable with the present invention are as follows:
Birnaviruses: infectious pancreatic necrosis virus, infectious bursal disease virus.
Reoviruses: reovirus, rice dwarf virus.

Shell proteins from virus particles that package DNA can also be employed. These are constructed in the same way, by expression in cells using plasmids that drive the synthesis of both the shell protein and pieces of DNA (usually single-stranded) that associate with the protein and get packaged inside. Exemplary DNA viruses having assemblable shell proteins employable with the present invention are as follows:
Parvoviruses: adeno-associated virus, canine parvovirus, feline panleukopenia virus, porcine parvovirus.
Microviruses: bacteriophages phi-x 174, G4, alpha-3.
Podoviruses: bacteriophages P22, T7, epsilon 15.
Polyomavirus: SV40, Murine polyomavirus, Merkel cell virus.

Non-Viral Shell Proteins

Exemplary non-viral shell proteins that are assemblable to produce protein nano-particles employable with the present invention are as follows: lumazine synthase, ferritin, carboxysomes, encapsulin, vault proteins, GroEL, and heat shock proteins.

Capsid-Polynucleotide Binding Sequences Are Well Understood and Predictable

Viral polynucleotides play an important role in viral assembly by their interaction with the inner surface of capsid proteins. The structural and functional role of RNA in the assembly process of virus particles is well studied and understood, see for example, A. Schneemann, Annu. Rev. Microbial. 2006, 60, 51-67. The binding of specific RNA elements binding to particle shell proteins has been extensively studied. See for example, G. G. Pickett, et al., Nucleic Acids Research, 1993 21(19), 4621-4626; D. S Peabody, D. S., et al., Nucleic Acids Research, 2002, 30(19), 4138-44; and G. W. Witherell et al., Biochemistry 1989, 28, 71-76 71. Capsid-polynucleotide binding sequences and interactions are well known and predictable.

Association with Capsid Inner Surface By Polynucleotide Having Aptameric Subsequence

The binding site on Q-beta coat protein by viral RNA is not a continuous feature. Ten mutations have been identified that are important for RNA binding, viz., residues #32, 49, 56, 59, 61, 63, 65, 89, 91, 95 of Q-beta coat protein. See: F. Lim, et al., The Journal of biological chemistry, 1996 271(50), 31839-45.

The use of the Qbeta “hairpin” RNA as a subsequence in bifunctional polypeptide SEQ ID 3 exploits a natural interaction. Similar RNA/capsid interactions are known for other viruses and bacteriophages and the relevant regions of the inner surface for RNA association have been identified. There are also many protein domains that are known to bind RNA or DNA.

Alternatively, the interaction can be engineered by appending or inserting a peptide tag to the shell protein with a sequence known to bind to a known aptamer, viz., a peptide tag/aptamer pair. The strategy is the same as employed for the peptide tag/aptamer pair employed with the cargo protein (see supra). The strategy is reliable and predictable. The strategy was exemplified and characterized in cells by F. Sieber et al., Nucleic acids research, 2011 39(14).

General Procedure: Using Protein-Packaged Enzymes

In general, the particle containing the desired enzyme(s) is incubated with the substrate and any necessary cofactors in standard buffer or buffer/organic solvent mixtures if required for the solubility of the small molecules. (Examples of organic solvent: methyl alcohol, dimethyl sulfoxide, N,N,-dimethyl formamide, and ethylene glycol. Others can also be used.)

The reaction can be performed at a range of temperatures up to the decomposition temperature of the protein shell or the denaturation temperature of the encapsulated enzyme; for the Qβ examples, the former temperature is approximately 85° C., and the latter varies with the enzyme.

Products are isolated away from protein by a variety of techniques, including extraction with organic solvent; filtration through size-exclusion columns or membranes, and high-performance liquid chromatography.

Nano-Particulate Catalytic System: Embodiments/Uses

The nano-particles of the invention can be used for the catalysis of chemical reactions in a variety of embodiments. For example, as discussed above, and exemplified below, the cargo enzyme can be a hydrolytic enzyme, catalyzing hydrolysis of amide or phosphate bonds. Other hydrolytic reactions can include esterase and lipase-catalyzed reactions acting on ester bonds, as well. In other embodiments, the hydrolytic reactions can be carried out by glycosidase enzymes, specifically hydrolyzing glycosidic bonds such as in polysaccharides, glycosylated proteins, and the like. In various embodiments, the invention provides a method of catalyzing a reaction in solution, comprising contacting a reaction-starting material with the catalytic nano-particle of the invention or a catalytic nano-particle prepared by a method of the invention, in solution, under conditions suitable for the reaction to occur.

In various embodiments, a nano-particle incorporating multiple types of enzymes can be used to carry out a series of reactions. In various embodiments, mixtures of various types of nano-particles of the invention, each nano-particle itself comprising one or more types of enzymes, can be used to carry out a series of reactions. For example, biosynthetic reactions resulting in a biocatalytic synthesis of a natural product or analog thereof, e.g., an antibiotic, an alkaloid, a hormone, or the like.

The particulate nature of the catalyst offers significant advantages in chemical processing. Reactions that are carried out in solution using the nano-particles in catalytic form are amenable to facile removal of the catalyst at the completion of the reaction. The solid nano-particles can be removed from the reaction solution by ultrafiltration, centrifugation, or similar techniques, leaving the reaction products in clean form in the reaction solvent.

In various embodiments, the solution is a substantially aqueous solution. When the reaction solvent is water, the reaction substrates necessarily have at least moderate water solubility, such that dissolved molecules of the substrate can diffuse into the protein nano-particle interior and contact the catalytic enzymes. Examples of such at least moderately water-soluble reaction substrates include peptides, proteins, saccharides, and water-soluble small molecules.

In various embodiments, substrates may have poor water solubility, and the addition of an organic solvent to the reaction medium must be used to dissolve the reaction substrate to a sufficient degree to allow a useful concentration in the reaction medium. The organic solvent should not cause degradation or denaturation of the cargo protein within the protein nano-particle. The inventors herein believe that the enhanced stability of cargo enzymes compared to free enzymes in solution can extend to the presence of organic solvents. Examples of water-soluble organic solvents include lower alcohols such as ethanol, amides such as N,N-dimethylformamide and N-methyl-pyrrolidone, sulfoxides such as DMSO, and the like. The presence of organic cosolvents can make poorly water-soluble substrates accessible for catalytic transformation by the encapsidated enzymes of the invention.

In various embodiments, the catalytic nano-particles can be merely dispersed in solution, as described above. In other embodiments, the nano-particles can be immobilized, such as in a porous matrix, allowing permeation by a solvent with a dissolved reaction substrate therein. Again, the solvent can be water, or can further include organic solvents, such as water-soluble organic solvents. The reaction solution can be passed or pumped through the matrix in which the catalytic nano-particles are disposed, such that the reaction products are found in the solution that has passed through the matrix material.

Accordingly in various embodiments, the invention provides a nanostructured construct comprising a plurality of nano-particles of the invention or of nano-particles prepared by the method of the invention, disposed within a structured matrix. The nano-particles can be randomly dispersed in a matrix, such as silica gel, alumina, a zeolite, a porous organic polymer, or the like. Alternatively, the nano-particles can be assembled in an ordered array within a matrix wherein a plurality of ordered sites are provided, wherein each of the nano-particles is constrained in an energetically favored manner. For example, a nanocomposite comprising a crystalline or quasi-crystalline array of an inorganic or an organic material can be designed to accommodate a plurality of nano-particles of the invention. Such composite materials can be used as catalytic systems in a variety of embodiments, as are apparent to the person of skill in the art.

### EXAMPLES

Materials and Methods

Cloning:

Peptidase E (AP_{—}004522, NCBI) was first coded into a fusion with the Qβ coat protein in an attempt to create hybrid particles with enzymes expressed on the outer capsid surface. The PepE gene was amplified by PCR directly from One Shot Top10 (Invitrogen) E. coli with the primers pepE-F1 and pepE-R1 (Table S1). Overlap extension PCR with PepE gene PCR product and Qβ coat protein (CP) amplified with CP-F1 and CP-R1 resulted in CP fused to PepE through a 24 bp linker sequence, corresponding to amino acids GGASESGG. The fusion product was digested with NcoI and Xho, gel purified, and ligated into similarly digested pCDF-1b (Novagen) to make plasmid pCDF-CP-pepE. Dual expression of this plasmid and the corresponding plasmid coding for the coat protein alone gave rise to hybrid particles as previously described for other fusion domains. However, the unstable nature of the hybrid protein nano-particles produced in this case proved difficult to purify and were not pursued.

TABLE S1
PepE and Qβ coat protein primers used for production of a fused
coat protein construct, as well as encapsidated proteins. Overlap
sequences are noted in italics.

| Primer Name | Primer Sequence |
|---|---|
| pepE-F1 | 5′-cgcgagcgaaagcggcggtatggaactgcttttattgagtaa-3′ |
| | [SEQ ID No: 34] |
| pepE-R1 | 5′-aagctggtcaccgtttttaactcgagcgg-3′ |
| | [SEQ ID No: 35] |
| CP-F1 | 5′-catgccatggcaaaattagagactgttact-3′ |
| | [SEQ ID No: 36] |
| CP-R1 | 5′-cgctttcgctcgcgccaccatacgctgggttcagct-3′ |
| | [SEQ ID No: 37] |
| pepE-F2 | 5′-catgccatggaactgcttttattgagtaa-3′ |
| | [SEQ ID No: 38] |
| pepE-his-F1 | 5′-catgccatggcacatcaccaccaccatcac |
| | atggaactgcttttattgagtaac-3′ |
| | [SEQ ID No: 39] |
| Luc-F2 | 5′-catgccatggaagacgccaaaaac-3′ |
| | [SEQ ID No: 40] |
| Luc-R1 | 5′-gcggaaagtccaaattgtaactcgagcgg-3′ |
| | [SEQ ID No: 41] |
| Luc-E354K-F1 | 5′-ctattctgattacacccaaaggggatgataaac-3′ |
| | [SEQ ID No: 42] |
| Luc-E354K-R1 | 5′-gtttatcatcccctttgggtgtaatcagaatag-3′ |
| | [SEQ ID No: 43] |

The Rev-pepE fusion was prepared as follows. The pepE gene was amplified by PCR from the pCDF-CP-pepE coding plasmid with primers pepE-F2 and pepE-R1, digested with NcoI and XhoI, gel purified and ligated into a similarly digested pCDF vector coding for the synthetic Rev-peptide in-frame and directly upstream from the NcoI site. For free PepE, amplification by PCR from the CP-pepE coding plasmid was performed with forward primer pepE-his-F1 (Table S1: sequence in bold corresponds to hexahistidine motif) and pepE-R1. Resulting fragment was again digested and ligated into a similarly digested pCDF-1b vector, creating pCDF-pepE. Rev-pepE S120A was created by using site-selective mutagenesis with primers pepE-S120A-F1 and pepE-S120A-R1 to replace the active-site serine with alanine. This was fused to the Rev-peptide in the same manner as above. For Rev-luciferase, firefly luciferase was amplified by PCR from pRevTRE-Luc (Clontech) with primers Luc-F2 and Luc-R1 (Table S1). Resulting fragment was fused to the plasmid-encoded Rev peptide in the same manner as for Rev-pepE. The thermal-stable luciferase was generated by site-selective mutagenesis PCR using primers Luc-E354K-F1 and Luc-E354K-R1 to replace the glutamate at position 354 with a lysine. This was amplified and fused to Rev in the same manner as WT luciferase.

All sequences were verified by direct sequencing of forward and reverse strands using unique primers at either ends (Retrogen). Plasmids were propagated in DH5a cells (BioPioneer) or One Shot Top10 (Invitrogen) and grown in SOB (Difco).

Protein Nano-Particle Production

E. coli BL21 (DE3) (Invitrogen) cells harboring the appropriate plasmids were grown in either SOB (Difco or Amresco) or MEM2 supplemented with carbenicilin, kanaymycin, or spectinomycin at 50, 100, and 100 μg/mL, respectively. Starter cultures were grown overnight at 37° C., and were used to inoculate larger cultures. Induction was performed with 1 mM IPTG at an OD600 of 1.0 in SOB or 2.0 in MEM for 4 hours at 37° C. for all PepE constructs, or 16 hours at 30° C. for luciferase constructs. Cells were harvested by centrifugation in a JA-17 rotor at 10K RPM and were either processed immediately or stored as a pellet at −80° C. The cell lysate was prepared by resuspending the cell pellet with 5 mL Qβ buffer (20 mM Tris-HCl, pH 7.5, containing 10 mM MgCl2) or TBS and sonicating at 30 W for 3 minutes with 5-second bursts and 5-second intervals. Cell debris was pelleted in a JA-17 rotor at 14K RPM and 2M ammonium sulfate was added to the supernatant to precipitate the protein nano-particles. These were pelleted and resuspended in 0.5 mL of Qβ buffer or TBS. Lipids and membrane proteins were then extracted from particles with 1:1 n-butanol:chloroform; protein nano-particles remain in the aqueous layer. Crude protein nano-particles were further purified by sucrose density ultracentrifugation (10-40% w:v). Particles were either precipitated from the sucrose solution with 10% w:v PEG8000 or pelleted out by ultracentrifugation in a 70.1 Ti rotor (Beckman) at 70K RPM for at least 2 hours. After assessment of purity as described below, additional sucrose gradients were used to further purify protein nano-particles to >95%, if necessary.

The catalytic activity of Qβ@(RevPepE)n particles was unaffected by organic extraction, but the catalytic activity of Qβ@(RevLuc)n particles was sensitive to such treatment. Therefore, the organic extraction step was omitted for the luciferase particles. No change in the number of proteins packaged per protein nano-particle was observed between samples that were extracted and those that were instead subjected to many rounds of sucrose gradient ultracentrifugation.

Protein Nano-Particle Purification/Characterization

Purity and Quantitation of Encapsidated Proteins

The purity of assembled protein nano-particles was assessed by isocratic size-exclusion chromatography with a Superose 6 column on an Akta Explorer FPLC instrument. Non-aggregated Qβ particles elute approximately 3 mL after the void volume-associated peaks.

The protein content of each sample was analyzed with a Bioanalyzer 2100 Protein 80 microfluidics chip. The average number of encapsidated proteins was determined by normalizing the area integration of coat protein and cargo protein peaks to the calculated molecular weight of the proteins they signified, determining the molar ratio of coat protein to cargo protein and multiplying by 180 to obtain the number of cargo proteins loaded per protein nano-particle. Overall protein concentration was determined with Coomassie Plus Protein Reagant (Pierce) according to the manufacturer's instructions.

Electron Microscopy

TEM images were acquired with a HP CM100 electron microscope (HP) with 80 kV, 1 s exposure and Kodak 50163 film on carbon formavor grids stained with 2% uranyl acetate.

Dynamic Light Scattering

Purified particles were analyzed on a light-scattering plate reader (Wyatt Dynapro).

Analytical Ultracentrifugation (AUC)

Sedimentation velocity experiments were performed on a Beckman XL-1 analytical ultracentrifuge, using both absorbance (260 nm) and interference optics, giving the data shown in Figure S2. Experiments were run at 15,000 RPM at 25° C., after a one-hour equilibration period. Data were fit to a “continuous species model” with Sedfit.₃ Estimated molecular weight of each protein nano-particle was obtained by assuming WT protein nano-particles package the same amount of RNA as the infectious virion (4200 nucleotides ssRNA). Protein nano-particles packaging an enzyme were estimated to package≈80% the amount of RNA of WT based on spectroscopic measurement at 260 nm of equal amounts of protein. Estimated molecular weight: WT(empty) and Qβ@(RevLuc)₄=3.8 MDa; Qβ@(RevPepE)₁₈=4.0 MDa. This corresponds to the differences of peak density constants obtained from AUC: WT(empty)=76 S; Qβ@(RevLuc)₄=79 S, and Qβ@(RevPepE)₁₈=86 S. Infectious virions, which package the RNA genome and infection-related proteins were calculated to have a density constant of 84 S. This suggests that the calculated densities are in the correct range of values and that we are able to significantly increase this density with our RNA-directed protein packaging system.

Free pepE Production and Purification

The conditions used for expression of free PepE were the same as used for the protein nano-particles. To isolate the desired material, the cleared cell lysate was passed through a cobalt-NTA Talon resin column (0.5 mL bed volume). The column was washed with 3 column volumes of T buffer (20 mM Tris-HCl pH 7.5), 3 volumes of T+20 mM imidazole, 2 volumes of T+100 mM imidazole and eluted with T+300 mM imidazole. Fractions containing PepE were pooled and dialyzed against two changes of 2 L of T and concentrated with an Amicon Ultra centrifugal filtration unit (10 kDa MWCO, Millipore). Purity was assayed by chip-based electrophoresis as above.

Enzymatic Activity

All experiments were run in triplicate and all runs with encapsidated enzyme were performed in parallel with purified free enzyme for comparison. All assays were performed with respect to overall enzyme concentration, not total protein concentration.

Peptidase E activity and kinetics were analyzed with fluorescent substrate aspartate-4-amino-7-methyl-coumarin (Asp-AMC) (Bachem), using a Thermo Varioskan Flash plate reader (excitation 352 nm, emission 438 nm, 5 nm slit, 100 ms read time). For determinations of kinetic parameters, 95 μL of 0-0.8 mM substrate in PBS buffer was added to 5 μL of a 20× enzyme solution of His6-PepE or Qβ@(Rev-PepE) and read immediately. Protein nano-particles that packaged an active-site knockout mutant (S120A) of pepE displayed no cleavage of the substrate.

For thermal protection studies, 60 μL of a 4.0 μg/mL solution (PepE concentration, in PBS) was incubated at the indicated temperature for 30 minutes. The solutions were then allowed to equilibrate to room temperature for 30 minutes before 50 μl was added to 50 μl of the substrate (0.6 mM final concentration). Initial velocities for every incubation temperature were normalized to the initial velocity of the free or packaged pepE incubated at 4° C. To determine the thermal half-life of the enzymes, the assay was the same as above, except the temperature was maintained at either 45° C. or 50° C. At the time point specified, 60 μL were taken and maintained at 4° C. until the end of the experiment. All samples were equilibrated to room temperature for 30 minutes and activity was assayed as described above. Initial velocities were normalized to activity at the t=0 time point for either 45° C. or 50° C. temperature. Activity measurements vs. time were plotted; an exponential decay non-linear fit was used to obtain half-life values.

For protease protection studies, 0.2 mg of Proteinase K (Invitrogen, >20 U/mg) was added to 150 μL of a 0.04 mg/mL His6-pepE or Qβ@(RevPepE)9 (in PBS) and incubated at room temperature. At the time points indicated, 5 μL aliquots were taken and 95 μL of substrate (0.76 mM final concentration Asp-AMC) was added and initial rates were measured. All data points were normalized to control treatments where proteinase K was not added. WT Qβ nano-particles were added to the His6-pepE samples to make the total protein concentrations in both samples equal.

Luciferase activity and kinetics were assayed by measuring the intensity of luminescence induced with D-luciferin (Anaspec, Inc.) in the plate reader. Purified luciferase (U.S. Biological) was reconstituted as recommended by the manufacturer and aliquots were stored at −80° C. and thawed immediately before use. Km values for D-luciferin and ATP were identified using a range of concentrations of each substrate (0-2 mM and 0-3 mM, respectively) in 30 mM HEPES pH 7.5 with 15 mM MgSO4, 0.16 nM enzyme (final concentrations). Activity was initiated by injecting 50 μL of a 2× enzyme solution into 50 μL luciferin or ATP of varying concentrations with all other components. Luminescence was measured immediately for 10 seconds. This emission intensity was plotted vs. substrate concentration of the varied reagent. A Michaelis-Menten non-linear fit was used to obtain Km,app and Vmax values. For time course measurements, luminescence was measured for 1 second every 2 minutes.

For luciferase, absolute kcat values are difficult to determine because the conversion between light output and number of catalytic turnovers is not clearly quantified. However, values of kcat could be calculated by assuming that the output light intensity at saturation represents Vmax of free luciferase in all experiments. Saturating relative light units can then be converted to turnovers by converting the specific activity of the enzyme into molecules of pyrophosphate released.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that, although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### DETAILED DESCRIPTION OF DRAWINGS

FIG. 1 illustrates a scheme for packaged molecular machines employing dual expression vectors that guide the preparation of Qβ virus-like particles encapsulating multiple enzymes. Packaging is promoted by RNA aptamer sequences that bridge between the coat protein and a peptide tag fused to the desired cargo (see scheme). Peptidase E and luciferase were encapsulated and shown to be catalytically active inside the protein nano-particle.

FIG. 2 illustrates a schematic representation of the technique used to package protein inside Qβ protein nano-particles. Dual-plasmid transformation of E. coli with compatible T7 expression vectors is the only input into the system. IPTG induction results in the expression of capsid protein (CP), Rev-tagged cargo enzyme, and bifunctional RNA. The Rev-tag binds to the -Rev aptamer (apt), and Qβ genome packaging hairpin (hp) binds to the interior of the CP monomers, thus tethering the enzyme to the interior of the protein nano-particle with the coat protein (cp) RNA sequence acting as the linker.

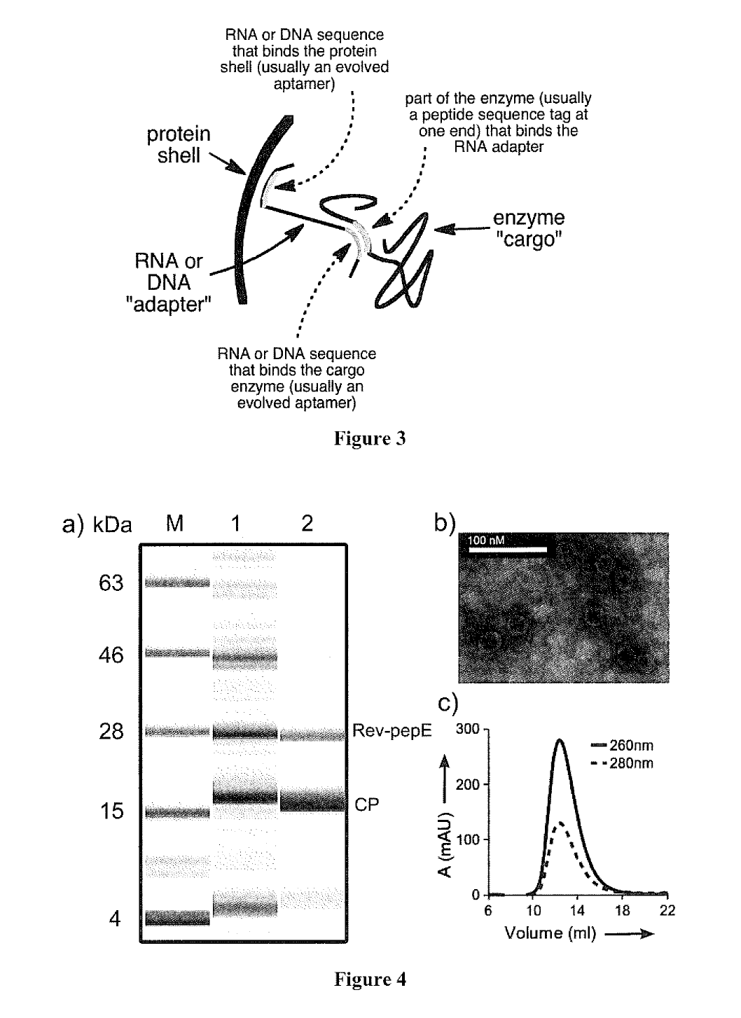

FIG. 3 illustrates an enlarged detail of the tri-molecular construct comprising a protein shell and a cargo protein linked by a bifunctional polynucleotide.

FIGS. 4 (A), (B) and (C) illustrate the physical characterization of Qβ@(RevPepE)18: (A) illustrates an electrophoretic analysis: lane M=protein ladder marker; 1=E. coli cell lysate 4 h after induction; 2=purified particles showing CP and Rev-pepE bands. (B) illustrates transmission electron micrograph; images are indistinguishable from those of WT Qβ protein nano-particles. (C) illustrates size-exclusion FPLC (Superose 6) showing intact nature of particles.

FIGS. 5 (A) and (B) illustrate the kinetics of PepE-catalyzed hydrolysis of fluorogenic Asp-AMC. Squares show the average of three independent initial rate measurements (<4 min.) with standard deviation as the error bars. Solid curves show the best fit using the Michaelis-Menten equation, giving the parameters shown.

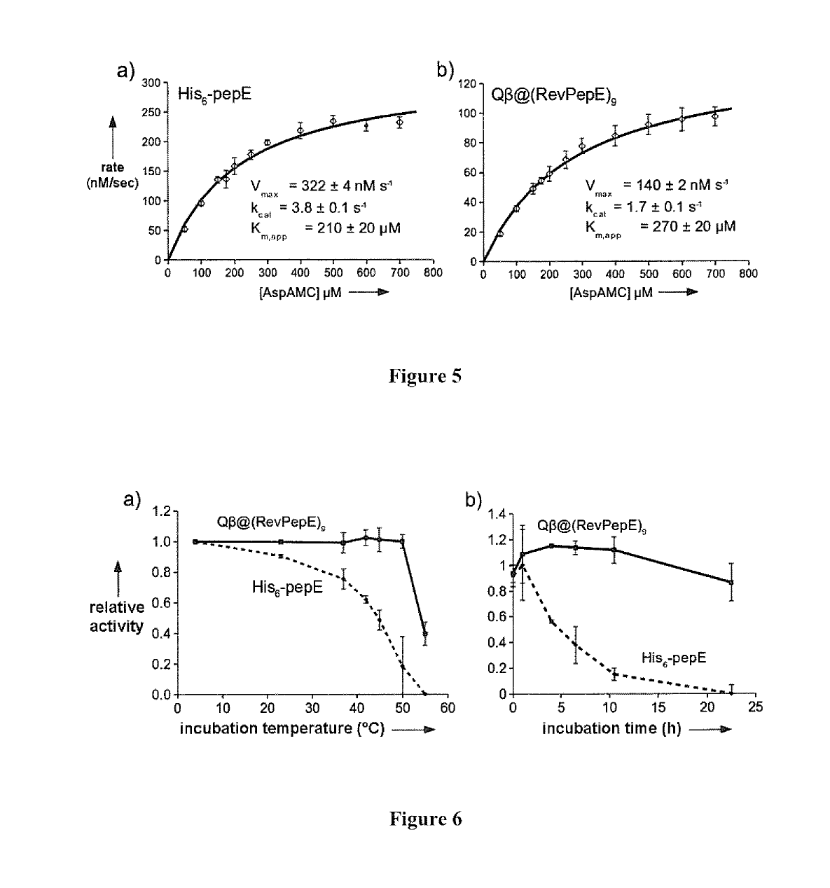

FIGS. 6 (A) and (B) illustrate the protection from thermal and protease inactivation of peptidase E by encapsidation. (A) illustrates the relative initial (<10 min.) rates of substrate hydrolysis after incubation of the enzyme for 30 minutes at the indicated temperature followed by cooling to room temperature before assay. The rate exhibited by enzyme incubated at 4° C. was set at 100%. (B) illustrates the telative initial rates of substrate hydrolysis after incubation at specified time with proteinase K. Data is represented as a percentage of a buffer control at each time point. Points are averages of independent measurements in triplicate and error bars are the standard deviation.

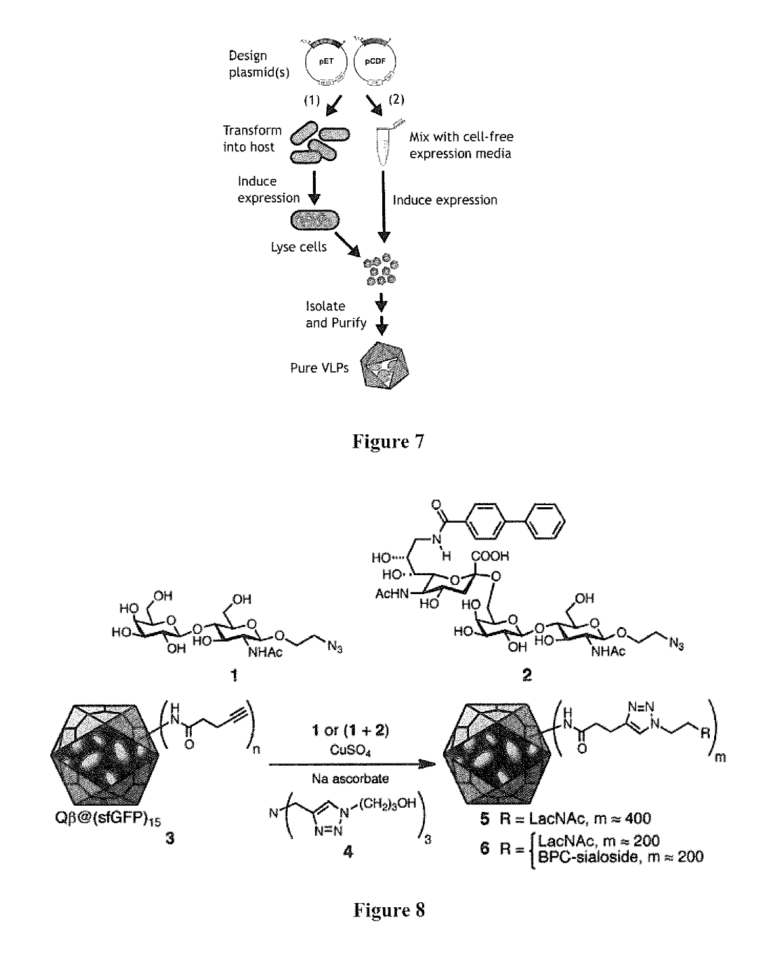

FIG. 7 illustrates cellular and cell-free pathways for producing protein. In the cellular pathway, a host cell, such as E. coli, other bacterial cells, yeast, algae, mammalian cells, insect cells, is transformed with one or more plasmids that code for the production of the desired shell protein, the desired cargo protein, and the desired trifunctional RNA adapter. The plasmids are preferably designed with inducible promoters that trigger the production of their respective components upon the addition of a molecule such as isopropyl beta-d-1-thiogalactopyranoside (IPTG), tetracycline, or arabinose; or a change in temperature; or other stimulus. The particles self-assemble in the expression cells and are isolated after the cells are broken open. The cellular process using yeast cells (without encapsulation of cargo proteins) was described by J. Freivalds, et al., Journal of Biotechnology 2006, 123 (3), 297-303. In contrast, cell-free expression may be employed using similar plasmids. This method also allows the addition of polynucleotides to be packaged, when such polynucleotides have been produced separately. See, for example, two papers that describe a method of cell-free expression which has been used to make virus-like particles: M. C. Jewett, et al., Biotechnol. Bioeng. 2004, 86, 19-26; and B. C. Bundy, et al., Biotechnol. Bioeng. 2008, 100, 28-37.

FIG. 8 illustrates synthetic schemes for the derivatization of Qβ@GFP15 with glycan ligands LacNAc (using 1) and the BPC derivative of sialic acid (using 2) by Cu-catalyzed azide-alkyne cycloaddition chemistry. It has previously been demonstrated that conjugation of the 9-biphenylcarbonyl (BPC) derivative of the sialoside Siaα2-6Galβ1-4GlcNAc(2) (see N. R. Zaccai, et al., Structure 2003, 11, 557-567; and B. E. Collins, et al., J. Immunol. 2006, 177, 2994-3003, and references therein) to Qβ endows the particle with strong and selective affinity for cells bearing the lectin CD22 (E. Kaltgrad, et al., J. Am. Chem. Soc. 2008, 130, 4578-9). To demonstrate the practicality of packaged fluorescent proteins for tracking such particles, Qβ@sfGFP (W. Xu, et al., Proc. Natl. Acad. Sci. U.S.A. 1996, 93, 7475-7480) was decorated with a short alkyne linker by acylation of surface amino groups (giving 3). The resulting f4 particle was addressed by Cu-catalyzed azide-alkyne cyclo-addition (CuAAC) under the influence of the accelerating ligand 4 (V. Hong, et al., Angew. Chem., Int. Ed. 2009, 48, 9879-9883). The azide component was either the Galβ1-4GlcNAc (LacNAc) disaccharide azide (1) alone as a negative control or a 1:1 mixture of 1 and 2 at the same overall concentration. In this way, the resulting particles 5 and 6 bore identical numbers of triazole-linked glycans, but only one (6) displayed the high-affinity CD22 ligand. MALDI-MS analysis showed coat protein subunits bearing 0, 1, 2, and 3 glycans. Estimation of their relative amounts (M. K. Patel, et al., Chem. Commun. 2010, 46, 9119-9121) indicated an average loading of 400 glycans per particle.

### DEFINITIONS

Capsule: For purposes of the present disclosure, the term “capsule” is defined herein to mean a nanoparticle sized structure having a well organized outer layer that defines an enclosure and serves to limit diffusion of large solutes from the exterior space into the enclosure. The enclosure is capable of containing something.

Synthetic Capsule Construct: For purposes of the present disclosure, the term “synthetic capsule construct” is defined herein to mean a non-naturally occurring capsule. Q-beta virus-like particles are exemplary synthetic capsule constructs.

Aptamer: For purposes of the present disclosure, the term “aptamer” is defined herein to mean an oligonucleotide having binding affinity and/or specificity for a protein tag or a protein binding site. The protein binding site may be either naturally occurring or evolved to have binding activity and includes zinc finger proteins. The aptamer may be either DNA or RNA. Also, it may be either naturally occurring or synthetic. When embedded as a subsequence within a longer polynucleotide sequence, the aptamer substantially maintains its binding affinity. Aptamers have excellent molecular recognition properties. Synthetic aptamers are usually selected initially from a large random sequence pool of oligonucleotides and then further evolved or engineered through repeated rounds of in vitro selection or by systematic evolution of ligands by exponential enrichment (SELEX) to bind to the desired peptide or protein target. For purposes of the present disclosure, the aptamer, after being evolved as an oligonucleotide to achieve the desired binding properties, is then embedded into a multifunctional polynucleotide in such a manner as to substantially preserve its binding activity as an oligonucleotide.

Aptameric Activity: For purposes of the present disclosure, the term “aptameric activity” is defined herein to mean an activity having the nature of an aptamer. When an aptamer is embedded as a subsequence within a longer polynucleotide sequence, the aptamer substantially maintains its binding affinity and imparts an aptameric activity to the polynucleotide. Aptamers have excellent molecular recognition properties. Synthetic aptamers are usually selected initially from a large random sequence pool of oligonucleotides and then further evolved or engineered through repeated rounds of in vitro selection or by systematic evolution of ligands by exponential enrichment (SELEX) to bind to the desired peptide or protein target. For purposes of the present disclosure, the aptamer, after being evolved as an oligonucleotide to achieve the desired binding properties, is then embedded into a multifunctional polynucleotide in such a manner as to substantially preserve its binding activity as an oligonucleotide. Alternatively, a polynucleotide may be evolved in the manner of an aptamer so as to impart aptameric activity to a subsequence within such polynucleotide or may achieve such activity without in vitro evolution, i.e., as a product of nature.

Shell: For purposes of the present disclosure, the term “shell” is defined herein to mean the outer layer of a capsule, including synthetic capsule constructs. As employed here, the term “shell” includes the outer layer of virus-like-particles (VLPs) and the outer layer of non-VLP nanoparticles having the characteristics of a capsule or synthetic capsule construct.

Shell protein: For purposes of the present disclosure, the term “shell protein” is defined herein to mean any protein or set of proteins capable of self-assembly or directed-assembly to form a “shell” of a capsule or synthetic capsule construct. Q-beta capsid proteins are exemplary shell proteins. More generally, shell proteins may be either viral or non-viral. Viral shell proteins include shell proteins originating from bacteriophages. Viral shell proteins are structural proteins expressed by either RNA or DNA viruses or bacteriophages. The RNA or DNA viruses or bacteriophages may naturally contain either single-stranded or double-stranded RNA or DNA. Viral shell proteins include capsid proteins, coat proteins, and envelope proteins and are capable of self-assembly to form virus-like particles (VLPs) or protein nanoparticles (PNPs).

Shell protein receptor site: For purposes of the present disclosure, the term “shell protein receptor site” is defined herein to mean a receptor site on the surface of a shell protein facing inwardly toward the enclosure defined by the capsule or synthetic capsule construct. The shell protein receptor site may be naturally occurring or non-naturally occurring. Preferred shell protein receptor sites are naturally occurring and, if they are viral in origin, specifically bind viral polynucleotides as part of the viral assembly process. The RNA-binding site of bacteriophage Q-beta coat protein described by F. Lim, et al., is a preferred shell protein receptor site. See, for example, J. Biol. Chem. 1996, 271 (50), 31839-45. Alternatively, a non-naturally occurring shell protein receptor site may be introduced by means of a tag.

Peptide tag: For purposes of the present disclosure, the term “peptide tag” is defined herein to mean a peptide sequence genetically grafted onto a recombinant protein, usually appended, for affording affinity to an aptamer. An exemplary protein tag is Rev, which has binding affinity for the aptamer, α-Rev.

Bifunctional polynucleotide: For purposes of the present disclosure, the term “bifunctional polynucleotide” is defined herein to mean a polynucleotide having two or more aptameric activities.

Address ligand: For purposes of the present disclosure, the term “address ligand” is defined herein to mean a ligand which, if conjugated to the outer surface of a capsule or synthetic capsule construct, affords such capsule or synthetic capsule construct an affinity or adhesion activity for binding a target.

Target: For purposes of the present disclosure, the term “target” is defined herein to mean a molecular structure spatially associated with a location to which it is desired to locate a synthetic capsule construct and/or the cargo protein therein, which molecular structure has an adhesion activity with respect to an address ligand.

Cargo protein: For purposes of the present disclosure, the term “cargo protein” is defined herein to mean any recombinant protein capable of being incorporated into a synthetic capsule construct. The cargo protein has either a protein tag or other binding site against which an aptamer has binding affinity.

1. A synthetic capsule construct for providing a protected chemical milieu, the construct comprising:
a shell having a plurality of shell proteins, said plurality of shell proteins being assembled with one another for forming said shell and defining an enclosure therein, each of said shell proteins, when assembled for forming said shell, having an interior surface facing inwardly toward said enclosure and an exterior surface facing outwardly away from said enclosure, said shell serving to restrict permeability to and from said enclosure for providing the protected chemical milieu therein, said shell proteins being recombinant;
a cargo protein, said cargo protein being recombinant and optionally including a peptide tag; and
a bifunctional polynucleotide having both a first aptameric activity for binding said cargo protein and a second aptameric activity for retaining said bifunctional polynucleotide within said enclosure by assembly with the interior surface of said shell protein,
said bifunctional polynucleotide being non-naturally occurring; said bifunctional polynucleotide serving to link said cargo protein within said enclosure for providing the said cargo protein with the protected chemical milieu therein.

2. The synthetic capsule construct of claim 1, wherein said cargo protein being selected from a group consisting of enzymes and signaling proteins.

3. The synthetic capsule construct of claim 2, wherein said cargo protein being selected from a group consisting of peptide cleavage enzymes, ester cleavage and formation enzymes, phosphate cleavage and formation enzymes, glycosyl transferases, alkylating enzymes, oxidases, reductases, dehydrating and hydrating enzymes, decarboxylases, carboxylases, aldolases, transaldolases, carbon-nitrogen lyases, nitrogen transferases, ammonia lyases, pyridoxal-requiring enzymes, isomerizing enzymes, prenyl group transferases, rearrangement enzymes, acetate, and priopionate fatty acid synthases.

4. The synthetic capsule construct of claim 1, wherein said cargo protein includes said peptide tag, said peptide tag being selected from a group consisting of peptide sequences genetically grafted onto the cargo protein and peptide sequences evolved within the cargo protein.

5. The synthetic capsule construct of claim 1, wherein said shell protein being selected from a group consisting of capsid proteins, coat proteins, and envelope proteins.

6. The synthetic capsule construct of claim 5, wherein said shell protein being Q-beta capsid protein.

7. The synthetic capsule construct of claim 1, wherein said shell protein being selected from a group consisting of shell proteins of a type derived from a single stranded RNA virus, shell proteins of a type derived from a double stranded RNA virus, and shell proteins of a type derived from a DNA virus.

8. The synthetic capsule construct of claim 7, wherein the single stranded RNA virus is selected from the group consisting of icosahedral virus, bromovirus, comoviruses, nodavirus, picornavirus, tombusviruses, levivirus, and tymovirus.

9. The synthetic capsule construct of claim 7, wherein the double stranded RNA virus is selected from the group consisting of birnavirus and reovirus.

10. The synthetic capsule construct of claim 7, wherein the double stranded DNA virus is selected from the group consisting of enterobacteria phage, parvovirus, microvirus, podovirus, and polyomavirus.

11. The synthetic capsule construct of claim 1, wherein said shell protein being a non-viral recombinant protein capable of self assembly to form a synthetic capsule construct.

12. The synthetic capsule construct of claim 11, wherein said shell protein is selected from the group consisting of lumazine synthase, ferritin, carboxysome, encapsulin, vault protein, GroEL, and heat shock protein.

13. The synthetic capsule construct of claim 1, wherein said bifunctional polynucleotide is selected from the group consisting of bifunctional polynucleotide RNAs and bifunctional polynucleotide DNAs.

14. The synthetic capsule construct of claim 13, wherein said bifunctional polynucleotide is transcribed RNA from a template selected from a group consisting of a plasmid and a genome.

15. The synthetic capsule construct of claim 13, wherein said second aptameric activity of said bifunctional polynucleotide having a binding activity with respect to an inner surface receptor site on said shell protein.

16. The synthetic capsule construct of claim 13, wherein said second aptameric activity of said bifunctional polynucleotide having non-specific binding affinity for the inner surface of the shell protein.

17. The synthetic capsule construct of claim 13, wherein said first aptameric activity having been grafted into said bifunctional polynucleotide as an aptamer evolved for binding activity with respect to said tag.

18. The synthetic capsule construct of claim 1 being capable of binding to a target, the construct further comprising: an address ligand conjugated to the exterior surface of said shell protein for binding the construct to the target.

19. A synthetic tri-molecular construct comprising:
a shell protein, said shell proteins being recombinant;
a cargo protein, said cargo protein being recombinant and optionally including a peptide tag; and
a bifunctional polynucleotide having both a first aptameric activity for binding said cargo protein and a second aptameric activity for binding said shell protein, said bifunctional polynucleotide being non-naturally occurring;
said bifunctional polynucleotide being linked both to said cargo protein and to said shell protein.

20. A synthetic bi-molecular shell construct capable of binding a cargo protein, the construct comprising:
a shell protein, said shell protein being recombinant; and
a bifunctional polynucleotide having both a first aptameric activity for binding the cargo protein and a second aptameric activity for binding said shell protein, said bifunctional polynucleotide being non-naturally occurring;
said bifunctional polynucleotide being linked to said shell protein and being capable of linking to the cargo protein.

21. A synthetic bi-molecular cargo construct capable of binding a shell protein, the construct comprising:
a cargo protein, said cargo protein being recombinant and optionally including a peptide tag; and
a bifunctional polynucleotide having both a first aptameric activity for binding said cargo protein and a second aptameric activity for binding the shell protein, said bifunctional polynucleotide being non-naturally occurring;
said bifunctional polynucleotide being linked to said cargo protein and being capable of linking to the shell protein.

22. A process for assembling a synthetic capsule construct, the process comprising the following step:
combining a plurality of shell proteins together with one or more cargo proteins in the presence of one or more bifunctional polynucleotides under conditions for assembling the synthetic capsule construct,
the shell proteins being assembled with one another for forming a shell and defining an enclosure therein, each of the shell proteins, when assembled for forming the shell, having an interior surface facing inwardly toward said enclosure and an exterior surface facing outwardly away from the enclosure, the shell proteins being recombinant;
the cargo protein being recombinant and optionally including a peptide tag; and
the bifunctional polynucleotide having both a first aptameric activity for binding the cargo protein and a second aptameric activity for retaining the bifunctional polynucleotide within the enclosure by assembly with the interior surface of the shell protein, the bifunctional polynucleotide being non-naturally occurring; and
linking the bifunctional polynucleotide to the cargo protein for retaining the cargo protein within the enclosure of the synthetic capsule construct.

23. The process of claim 22, wherein said cargo protein is selected from a group consisting of peptide cleavage enzymes, ester cleavage and formation enzymes, phosphate cleavage and formation enzymes, glycosyl transferases, alkylating enzymes, oxidases, reductases, dehydrating and hydrating enzymes, decarboxylases, carboxylases, aldolases, transaldolases, carbon-nitrogen lyases, nitrogen transferases, ammonia lyases, pyridoxal-requiring enzymes, isomerizing enzymes, prenyl group transferases, rearrangement enzymes, acetate, and priopionate fatty acid synthases.

24. The process of claim 23, wherein said combination and linking steps occur within a host cell containing one or more plasmids encoding the shell proteins, the cargo proteins, and the bifunctional polynucleotides.

25. The process of claim 23, wherein said combination step occurs extra-cellularly under in vitro conditions.

26. The process of claim 23 comprising the further step of: conjugating an address ligand to the exterior surface of one or more the shell proteins.

27. A process for protecting a cargo protein from a solute, the process comprising the steps of:
confining a cargo protein within the enclosure of a synthetic capsule construct by linkage with a bifunctional polynucleotide, the synthetic capsule construct being of a type affording protection from the solute; and then
exposing the synthetic capsule construct to the solute; whereby the cargo protein is protected from the solute by enclosure within the synthetic capsule construct.

28. The process of claim 27 further comprising the steps of:
conjugating an address ligand to the synthetic capsule construct, the address ligand having binding activity with respect to a target having an adhesion activity with respect to the address ligand; and then
binding the synthetic capsule construct to a target by adhesion to the address ligand;
whereby the cargo protein becomes located adjacent to the target adhesion to the address ligand conjugated to the synthetic capsule construct.

29. The process of claim 27, wherein said cargo protein is selected from a group consisting of peptide cleavage enzymes, ester cleavage and formation enzymes, phosphate cleavage and formation enzymes, glycosyl transferases, alkylating enzymes, oxidases, reductases, dehydrating and hydrating enzymes, decarboxylases, carboxylases, aldolases, transaldolases, carbon-nitrogen lyases, nitrogen transferases, ammonia lyases, pyridoxal-requiring enzymes, isomerizing enzymes, prenyl group transferases, rearrangement enzymes, acetate, and priopionate fatty acid synthases.

30. A host cell for producing a synthetic capsule construct, the host cell comprising a first polynucleotide expressible for producing a recombinant cargo protein, a second polynucleotide expressible for producing a recombinant shell protein, and a third polynucleotide transcribable for producing a bifunctional polynucleotide capable of linking said recombinant shell proteins to said recombinant cargo proteins for assembly into a synthetic capsule construct, said first, second, and third polynucleotides being embedded in one or more potentially overlapping polynucleotides selected from a group consisting of plasmid polynucleotides and genomic polynucleotides.
